(19) United States
(12) Patent Application Publication
(10) Pub. No.: US 2025/0346635 A1
(43) Pub. Date: Nov. 13, 2025

(54) VASCULAR ENDOTHELIAL GROWTH FACTOR RECEPTOR-1 (VEGFR-1) INHIBITORS FOR PROMOTING MYELINATION AND NEUROPROTECTION

(71) Applicants: ICM (INSTITUT DU CERVEAU ET DE LA MOELLE ÉPINIÉRE), Paris (FR); INSERM (INSTITUT NATIONAL DE LA SANTÉ ET DE LA RECHERCH MÉDICALE), Paris (FR); CENTRE NATIONAL DE LA RECHERCHE SCIENTIFIQUE, Paris (FR); APHP (ASSISTANCE PUBLIQUE - HÔPITAUX DE PARIS), Paris (FR); SORBONNE UNIVERSITE, Paris (FR)

(72) Inventors: Brahim NAIT OUMESMAR, Paris (FR); Nadjet GACEM, Paris (FR); Cyril DEBOUX, Paris (FR)

(73) Assignees: ICM (INSTITUT DU CERVEAU ET DE LA MOELLE ÉPINIÉRE), Paris (FR); INSERM (INSTITUT NATIONAL DE LA SANTÉ ET DE LA RECHERCH MÉDICALE), Paris (FR); CENTRE NATIONAL DE LA RECHERCHE SCIENTIFIQUE, Paris (FR); APHP (ASSISTANCE PUBLIQUE - HÔPITAUX DE PARIS), Paris (FR); SORBONNE UNIVERSITE, Paris (FR)

(21) Appl. No.: 18/879,943

(22) PCT Filed: Jun. 30, 2023
(86) PCT No.: PCT/EP2023/068085

§ 371 Date: Dec. 30, 2024

(30) Foreign Application Priority Data
Jun. 30, 2022 (EP) 22305962.7

(51) Int. Cl. C07K 14/00; A61K 38/00; C12N 15/113

(52) U.S. Cl. CPC C07K 14/001; C12N 15/1136; A61K 38/00; C12N 2310/14; C12N 2310/531

(57) ABSTRACT

Vascular endothelial growth factor receptors (VEGFRs) inhibitors, in particular VEGFR-1 inhibitors, for use in promoting myelination and/or neuroprotection in a subject. Such VEGR-1 inhibitors include small organic molecules, peptides, antibodies, antibody fragments, antibody mimetics, or nucleic acids. Also, compositions, pharmaceutical compositions, medicaments and kits of parts that include VEGFR-1 inhibitors and their use for promoting myelination and/or neuroprotection in a subject.

### FIELD OF INVENTION

The present invention relates to vascular endothelial growth factor receptors (VEGFRs) inhibitors, in particular VEGFR-1 inhibitors, for use in promoting myelination and neuroprotection in a subject. The present invention also relates to compositions, pharmaceutical compositions, medicaments and kits of parts comprising VEGFR-1 inhibitors and their use for promoting myelination and neuroprotection in a subject.

### BACKGROUND OF INVENTION

Myelin is an essential component of the nervous system and ensures life-long nervous system health and function. Indeed, the myelin sheath insulates the axon and facilitates saltatory conduction, which allows for rapid conduction of action potentials. Many neurological diseases are characterized by the destruction of myelin, i.e. a process called demyelination, which may ultimately lead to neuronal loss. Several causes of demyelination have been identified so far and include, for example, inflammatory processes, viral infections, acquired metabolic derangements, hypoxia-ischemia and traumatic injury.

Among the diseases associated with demyelination, multiple sclerosis (MS) is the first cause of non-traumatic disability in young adults. A recent study estimated that, in the United States, multiple sclerosis represents a cost of about $5.1 billion each year due to sick leave, premature retirement and loss of income. Thus, there is an urgent need to develop new therapies for treating central nervous system diseases associated with demyelination.

Inventors have found that VEGFRs inhibitors, and especially specific and selective inhibitors of VEGFR-1, can promote both myelination and neuroprotection, demonstrating therefore the promising use of VEGFR-1 inhibitors in remyelination therapy.

### SUMMARY

The present invention relates to an inhibitor of vascular endothelial growth factor receptor 1 (VEGFR-1) for use in promoting myelination and/or neuroprotection in a subject in need thereof.

In one embodiment, said inhibitor is selected from the group comprising or consisting of small organic molecules, peptides, antibodies, antibody fragments, antibody mimetics, and nucleic acids.

In one embodiment, said inhibitor is a small organic molecule. In one embodiment, said small organic molecule is selected from the group comprising or consisting of ZM306416 (CB 676475), SU14813, and salts, derivatives and combinations thereof.

In one embodiment, said inhibitor is a peptide. In one embodiment, said inhibitor is a peptide selected from the group comprising or consisting of peptides with SEQ ID NOs: 6 to 10 and combinations thereof, more preferably said inhibitor is a peptide with SEQ ID NO: 6.

In one embodiment, said inhibitor is a nucleic acid. In one embodiment, said inhibitor is a shRNA, preferably selected from the group comprising or consisting of shRNAs with SEQ ID NOs: 15 to 18, and combinations thereof.

In one embodiment, the subject is affected or diagnosed with a demyelinating disease.

In one embodiment, the demyelinating disease is selected from the group comprising or consisting of multiple sclerosis, optic neuritis, cerebral ischemia, leukodystrophies and traumatic brain injury (TBI).

In one embodiment, the demyelinating disease is multiple sclerosis. In one embodiment, the multiple sclerosis is selected from the group consisting of clinically isolated syndrome (CIS), relapsing remitting multiple sclerosis (RRMS), primary progressive multiple sclerosis (PPMS), and secondary progressive multiple sclerosis (SPMS), preferably the multiple sclerosis is a progressive form of the disease.

In one embodiment, said subject has received, is receiving or will receive an anti-inflammatory drug.

In one embodiment, the anti-inflammatory drug is an immunomodulator or an immunosuppressant. In one embodiment, the anti-inflammatory drug is selected from the group comprising or consisting of interferon beta, Glatiramer acetate, Fingolimod, Dimethyl fumarate, Diroximel fumarate, Teriflunomide, Siponimod, Cladribine, Natalizumab, Ocrelizumab, Alemtuzumab, Cyclophosphamide, Azathioprine, Mitoxandrone and combinations thereof.

The present invention also relates to a pharmaceutical composition comprising an inhibitor of VEGFR-1 as described hereinabove and at least one pharmaceutically acceptable excipient, for use in promoting myelination and/or neuroprotection in a subject in need thereof.

The present invention also relates to a kit of parts comprising, in one part, at least one inhibitor of VEGFR-1 and, in a second part, at least one anti-inflammatory drug.

In one embodiment, said kit of parts is for use in promoting myelination and/or neuroprotection in a subject in need thereof.

### Definitions

In the present invention, the following terms have the following meanings:

“About” preceding a value means plus or less 10% of said value.

“Adnectins”, also known as monobodies, is well known in the art and refers to proteins designed to bind with high affinity and specificity to antigens. They belong to the class of molecules collectively called “antibody mimetics”.

“Alphabody” that may also be referred to as Cell-Penetrating Alphabodies, refers to a type of antibody mimetics consisting of small 10 kDa proteins engineered to bind to a variety of antigens. Alphabodies are able to reach and bind to intracellular protein targets.

“Affibodies” refer to affinity proteins based on a 58 amino acid residue protein domain, derived from one of the IgG binding domain of staphylococcal protein A (Frejd & Kim, 2017. Exp Mol Med. 49(3):e306; U.S. Pat. No. 5,831,012).

“Affilins” refer to artificial proteins designed to selectively bind antigens. They resemble antibodies in their affinity and specificity to antigens but not in structure which makes them a type of antibody mimetic.

“Affitins” refer to highly stable engineered affinity proteins, originally derived from Sac7d and Sso7d, two 7 kDa DNA-binding polypeptides from Sulfolobus genera.

“Antibody” and “immunoglobulin”, as used herein, may be used interchangeably and refer to a protein having a combination of two heavy and two light chains whether or not it possesses any relevant specific immunoreactivity. “Antibodies” refers to such assemblies which have significant known specific immunoreactive activity to an antigen of interest (e.g., VEGFR-1). The term “anti-VEGFR-1 antibody” is used herein to refer to antibodies which exhibit immunological specificity for human VEGFR-1. As explained elsewhere herein, “specificity” for human VEGFR-1 does not exclude cross-reaction with species homologues of VEGFR-1, such as, for example, with simian VEGFR-1. Antibodies and immunoglobulins comprise light and heavy chains, with or without an interchain covalent linkage between them. Basic immunoglobulin structures in vertebrate systems are relatively well understood. The generic term “immunoglobulin” comprises five distinct classes of antibody that can be distinguished biochemically. Although the following discussion will generally be directed to the IgG class of immunoglobulin molecules, all five classes of antibodies are within the scope of the present invention. With regard to IgG, immunoglobulins comprise two identical light polypeptide chains of molecular weight of about 23 kDa, and two identical heavy chains of molecular weight of about 53-70 kDa. The four chains are joined by disulfide bonds in a “Y” configuration wherein the light chains bracket the heavy chains starting at the mouth of the “Y” and continuing through the variable region. The light chains of an antibody are classified as either kappa (κ) or lambda (λ). Each heavy chain class may be bonded with either a κ or λ light chain. In general, the light and heavy chains are covalently bonded to each other, and the “tail” regions of the two heavy chains are bonded to each other by covalent disulfide linkages or non-covalent linkages when the immunoglobulins are generated either by hybridomas, B cells or genetically engineered host cells. In the heavy chain, the amino acid sequences run from an N-terminus at the forked ends of the Y configuration to the C-terminus at the bottom of each chain. Those skilled in the art will appreciate that heavy chains are classified as gamma (γ), mu (μ), alpha (α), delta (δ) or epsilon (ε) with some subclasses among them (e.g., γ1-γ4). It is the nature of this chain that determines the “class” of the antibody as IgG, IgM, IgA IgD or IgE, respectively. The immunoglobulin subclasses or “isotypes” (e.g., IgG1, IgG2, IgG3, IgG4, IgA1, etc.) are well characterized and are known to confer functional specialization. Modified versions of each of these classes and isotypes are readily discernable to the skilled artisan in view of the instant disclosure and, accordingly, are within the scope of the present invention. As indicated above, the variable region of an antibody allows the antibody to selectively recognize and specifically bind epitopes on antigens. That is, the light chain variable domain (VL domain) and heavy chain variable domain (VH domain) of an antibody combine to form the variable region that defines a three-dimensional antigen binding site. This quaternary antibody structure forms the antigen binding site presents at the end of each arm of the “Y”. More specifically, the antigen binding site is defined by three complementarity determining regions (CDRs) on each of the VH and VL chains.

“Antibody fragment”, as used herein, refers to a part or region of an antibody which comprises fewer amino acid residues than the whole antibody. An “antibody fragment” binds antigen and/or competes with the whole antibody from which it derives for antigen binding (e.g., specific binding to VEGFR-1). Antibody fragments encompasses, without any limitation, a single chain antibody, a dimeric single chain antibody, a Fv, a scFv, a Fab, a Fab′, a Fab′-SH, a F(ab)′2, a Fd, a defucosylated antibody, a diabody, a triabody and a tetrabody. It may also encompass a unibody, a domain antibody, and a nanobody.

“Anticalins” refer to an antibody mimetic technology, wherein the binding specificity is derived from lipocalins. Anticalins may also be formatted as dual targeting protein, called Duocalins.

“Anti-inflammatory drug” refers to a drug or substance that reduces inflammation (redness, swelling, and pain) in the body.

“Armadillo repeat protein-based scaffold”, as used herein, refers to a type of antibody mimetics corresponding to artificial peptide binding scaffolds based on armadillo repeat proteins. Armadillo repeat proteins are characterized by an armadillo domain, composed of tandem armadillo repeats of approximately 42 amino acids, which mediates interactions with peptides or proteins.

“Atrimers” refer to binding molecules for target protein that trimerize as a perquisite for their biological activity. They are relatively large compared to other antibody mimetic scaffolds.

“Avimers” refer to an antibody mimetic technology.

“Diabodies”, as used herein, refer to small antibody fragments prepared by constructing scFv fragments with short linkers (about 5-10 residues) between the VH and VL such that inter-chain but not intra-chain pairing of the variable domains is achieved, resulting in a bivalent fragment, i.e., fragment having two antigen-binding sites. Bispecific diabodies are heterodimers of two “crossover” scFv fragments in which the VH and VL of the two antibodies are present on different polypeptide chains. Diabodies are described, for example, in patent EP0404097 or patent application WO1993011161.

“Demyelination”: refers to a pathologic process occurring in the nervous system in which the myelin sheath is damaged. The damage to the myelin sheath impairs the conduction of signals in the affected nerves. Consequently, the reduction in conduction ability causes deficiency in sensation, movement, cognition, and/or other functions depending on which nerves are involved. The demyelination might be due to genetics, infectious agents, autoimmune reactions, as well as unknown factors. The demyelination may affect the central nervous system (CNS) and/or the peripheral nervous system (PNS).

“Demyelinating diseases”: refers to diseases associated with demyelination (i.e. myelin loss).

“Derivative”: refers broadly to the modification or substitution of one or more chemical moieties on a parent compound and may include positional isomers, tautomers, zwitterions, enantiomers, diastereomers, racemates, isosteres or stereochemical mixtures thereof.

“Domain antibodies” refer to the smallest functional binding units of antibodies, corresponding to the variable regions of either the heavy or light chains of antibodies.

“Domain Kunitz peptide” refer to a type of antibody mimetics, and is based on the active domains of proteins inhibiting the function of proteases.

Evasins” refer to a class of chemokine-binding proteins.

“Fab” refers to fragment antibodies generated by papain digestion of whole IgG antibodies to remove the entire Fc fragment, including the hinge region. These antibodies are monovalent, containing only a single antigen binding site. In contrast, F(ab′)2 fragment antibodies are generated by pepsin digestion of whole IgG antibodies to remove most of the Fc region while leaving intact some of the hinge region. F(ab′)₂ fragments have two antigen-binding F(ab) portions linked together by disulfide bonds. The term “Fab′” refers to an antibody fragment having a molecular weight of about 50,000 and antigen binding activity, which is obtained by cutting a disulfide bond of the hinge region of the F(ab′)₂. Fab′-SH is the designation herein for Fab′ in which the cysteine residue(s) of the constant domains bear a free thiol group.

“Fynomers” refer to proteins that belong to the class of antibody mimetic. They are attractive binding molecules due to their high thermal stability and reduced immunogenicity.

“Fv”, as used herein, refers to the minimum antibody fragment that contains a complete antigen-recognition and -binding site. This fragment consists of a dimer of one VH and one VL in tight, non-covalent association. From the folding of these two domains emanate six hypervariable loops (three loops each from the heavy and light chain) that contribute to antigen binding and confer antigen binding specificity to the antibody. However, even a single variable domain (or half of an Fv comprising only three CDRs specific for an antigen) has the ability to recognize and bind antigen, although at a lower affinity than the entire binding site.

“Immunomodulator” refers to a substance that affects the functioning of the immune system.

“Immunosuppressant” refers to an agent that can suppress or prevent the immune response.

“Inhibitor of VEGFR-1” refers to a substance inducing an inhibition or down-regulation of a biological activity associated with activation of VEGFR-1, including any of the downstream biological effects otherwise resulting from the binding of the VEGFR-1 to its natural ligand.

“Knottin” (that may also be referred to as inhibitor cystine knot) refers to an antibody mimetic comprising a protein structural motif containing three disulfide bridges.

“Multiple sclerosis” or “MS” refers to an inflammatory demyelinating disease of the central nervous system (CNS).

“Neuroprotection” refers to the preservation of neuronal integrity and/or function in case of a nervous system injury, including for instance, demyelination, trauma, hypoxia-ischemia, oxidative stress and metabolic disturbances.

“Neuronal loss” refers to the loss of neuronal cells.

“Neurons” and “neuronal cells” are equivalent and can be interchanged in the present application.

“Nerves” and “nerve fibers” are equivalent and can be interchanged in the present application.

“Oligodendrocyte lineage cells”: as used herein, refers to the oligodendrocytes and oligodendrocyte progenitor cells (OPCs).

“Peptide”: refers to a linear polymer of amino acids of less than 50 amino acids linked together by peptide bonds.

“Promoting myelination” or “promoting myelin regeneration” refers to the process of promoting myelin formation by promoting survival, differentiation, and/or maturation of oligodendrocyte lineage cells and/or Schwann cells. In the context of the present invention, promoting myelination or myelin regeneration of neurons is equivalent to promoting myelination or myelin regeneration of nerve fibers.

“Salt”: refers to a chemical compound consisting of an ionic assembly of positively charged cations and negatively charged anions, which results in a compound with no net electric charge.

“Single chain antibody”, as used herein, refers to any antibody or fragment thereof that is a protein having a primary structure comprising or consisting of one uninterrupted sequence of contiguous amino acid residues, including without limitation (1) single-chain Fv molecules (scFv); (2) single chain proteins containing only one light chain variable domain, or a fragment thereof that contains the three CDRs of the light chain variable domain, without an associated heavy chain moiety; and (3) single chain proteins containing only one heavy chain variable region, or a fragment thereof containing the three CDRs of the heavy chain variable region, without an associated light chain moiety.

“Single-chain Fv”, also abbreviated as “sFv” or “scFv”, refers to antibody fragments that comprise the V_{H} and V_{L} antibody domains connected into a single amino acid chain. Preferably, the scFv amino acid sequence further comprises a peptide linker between the V_{H} and V_{L} domains that enables the scFv to form the desired structure for antigen binding.

“Selectivity” refers to the affinity of a molecule, such as an inhibitor, for VEGFR-1, which is at least 10-fold, 25-fold, 50-fold, 75-fold, 80-fold, 90-fold, 95 fold, 100-fold, 125-fold, 150-fold, 200-fold, 250-fold, 300-fold, 350-fold, 400-fold, 450-fold, or 500-fold higher than the affinity for other VEGFR, in particular VEGFR-2 and VEGFR-3.

“Small organic molecule” refers to a molecule of a size comparable to those organic molecules generally used in pharmaceuticals. The term excludes biological macromolecules (e.g., proteins, nucleic acids, etc.). Preferred small organic molecules range in size up to about 5000 Da, more preferably up to 2000 Da, and most preferably up to about 1000 Da. Preferred small organic molecules range in size up to about 5000 g/mol, more preferably up to 2000 g/mol, and most preferably up to about 1000 g/mol.

“Specificity” refers to the ability of a molecule, such as an inhibitor, to detectably bind its target, such as VEGFR-1, while having relatively little detectable reactivity with non-VEGFR-1 proteins. Specificity can be relatively determined by binding or competitive binding assays, using, e.g., Biacore instruments. Specificity can be exhibited by, e.g., an about 2:1, about 5:1, about 10:1, about 20:1, about 50:1, about 100:1, 10.000:1 or greater ratio of affinity/avidity in binding to the specific target versus nonspecific binding to other irrelevant molecules (in this case, the specific target is VEGFR-1).

“Subject” refers to a mammal, preferably a human. In one embodiment, a subject may be a “patient”, i.e. a warm-blooded animal, more preferably a human, who/which is awaiting the receipt of, or is receiving medical care or was/is/will be the object of a medical procedure, or is monitored for the development of a disease. In one embodiment, the subject is an adult (for example a subject above the age of 18). In another embodiment, the subject is a child (for example a subject below the age of 18). In one embodiment, the subject is a male. In another embodiment, the subject is a female.

“Schwann cells (SCs)” refer to the major glial cell type in the peripheral nervous system. They play essential roles in the myelination, development, maintenance, function and regeneration of peripheral nerves.

“Therapeutically effective amount” means the level or amount of agent that is aimed at, without causing significant negative or adverse side effects to the target, (1) delaying or preventing the onset of the demyelinating disease; (2) slowing down or stopping the progression, aggravation, or deterioration of one or more symptoms of the demyelinating disease; (3) bringing about ameliorations of the symptoms of demyelinating disease; (4) reducing the severity or incidence of the demyelinating disease; or (5) curing the demyelinating disease. A therapeutically effective amount may be administered prior to the onset of the demyelinating disease, for a prophylactic or preventive action. Alternatively, or additionally, the therapeutically effective amount may be administered after initiation of the demyelinating disease, for a therapeutic action or maintenance of a therapeutic action.

“Treating” or “treatment” or “alleviation” refers to both therapeutic treatment and prophylactic or preventative measures; wherein the object is to prevent or slow down (lessen) the targeted pathologic condition or disorder. Those in need of treatment include those already with the disorder as well as those prone to have the disorder or those in whom the disorder is to be prevented. A subject or mammal is successfully “treated” for the targeted pathologic disorder if, after receiving a therapeutic amount of the composition of the present invention, the patient shows observable effects on one or more of the followings; relief to some extent, of one or more of the symptoms associated with the specific disorder or condition; reduced morbidity and mortality, and improvement in quality of life issues. The above parameters for assessing successful treatment and improvement in the disorder are readily measurable by routine procedures familiar to a physician.

“VEGFR” or “Vascular endothelial growth factor receptor” refers to three related receptor tyrosine kinases binding to vascular endothelial growth factors (VEGFs): VEGFR-1 (also known as Flt1), VEGFR-2 (also termed KDR) and VEGFR-3 (also named Flt4). VEGFRs have seven extracellular immunoglobulin-like domains, a transmembrane domain and an intracellular tyrosine kinase domain. Ligands of VEGFR-1 are VEGFA, VEGFB and PLGF. VEGFR2 binds VEGFA and proteolytically processed VEGFC and VEGFD, while VEGFR3 is activated by the binding of VEGFC and VEGFD. An example of an amino sequence of human VEGFR-1 is SEQ ID NO: 1.

SEQ ID NO: 1
MVSYWDTGVLLCALLSCLLLTGSSSGSKLKDPELSLKGTQHIMQAGQTLH
LQCRGEAAHKWSLPEMVSKESERLSITKSACGRNGKQFCSTLTLNTAQAN
HTGFYSCKYLAVPTSKKKETESAIYIFISDTGRPFVEMYSEIPEIIHMTE
GRELVIPCRVTSPNITVTLKKFPLDTLIPDGKRIIWDSRKGFIISNATYK
EIGLLTCEATVNGHLYKTNYLTHRQTNTIIDVQISTPRPVKLLRGHTLVL
NCTATTPLNTRVQMTWSYPDEKNKRASVRRRIDQSNSHANIFYSVLTIDK
MQNKDKGLYTCRVRSGPSFKSVNTSVHIYDKAFITVKHRKQQVLETVAGK
RSYRLSMKVKAFPSPEVVWLKDGLPATEKSARYLTRGYSLIIKDVTEEDA
GNYTILLSIKQSNVFKNLTATLIVNVKPQIYEKAVSSFPDPALYPLGSRQ
ILTCTAYGIPQPTIKWFWHPCNHNHSEARCDFCSNNEESFILDADSNMGN
RIESITQRMAIIEGKNKMASTLVVADSRISGIYICIASNKVGTVGRNISF
YITDVPNGFHVNLEKMPTEGEDLKLSCTVNKFLYRDVTWILLRTVNNRTM
HYSISKQKMAITKEHSITLNLTIMNVSLQDSGTYACRARNVYTGEEILQK
KEITIRDQEAPYLLRNLSDHTVAISSSTTLDCHANGVPEPQITWFKNNHK
IQQEPGIILGPGSSTLFIERVTEEDEGVYHCKATNQKGSVESSAYLTVQG
TSDKSNLELITLTCTCVAATLFWLLLTLFIRKMKRSSSEIKTDYLSIIMD
PDEVPLDEQCERLPYDASKWEFARERLKLGKSLGRGAFGKVVQASAFGIK
KSPTCRTVAVKMLKEGATASEYKALMTELKILTHIGHHLNVVNLLGACTK
QGGPLMVIVEYCKYGNLSNYLKSKRDLFFLNKDAALHMEPKKEKMEPGLE
QGKKPRLDSVTSSESFASSGFQEDKSLSDVEEEEDSDGFYKEPITMEDLI
SYSFQVARGMEFLSSRKCIHRDLAARNILLSENNVVKICDFGLARDIYKN
PDYVRKGDTRLPLKWMAPESIFDKIYSTKSDVWSYGVLLWEIFSLGGSPY
PGVQMDEDFCSRLREGMRMRAPEYSTPEIYQIMLDCWHRDPKERPRFAEL
VEKLGDLLQANVQQDGKDYIPINAILTGNSGFTYSTPAFSEDFFKESISA
PKFNSGSSDDVRYVNAFKFMSLERIKTFEELLPNATSMEDDYQGDSSTLL
ASPMLKRFTWTDSKPKASLKIDLRVTSKSKESGLSDVSRPSFCHSSCGHV
SEGKRRFTYDHAELERKIACCSPPPDYNSVVLYSTPPI

“VEGFR function” or “Vascular endothelial growth factor receptor function” refers to a biological activity associated with activation of VEGFR, including any of the downstream biological effects otherwise resulting from the binding of the VEGFR to its natural ligand. VEGFR-1, VEGFR-2 or VEGF-3 function refers to a biological activity associated with activation of VEGFR-1, VEGFR-2 or VEGFR-3, respectively.

### DETAILED DESCRIPTION

The present invention relates to an inhibitor of vascular endothelial growth factor receptor 1 (VEGFR-1) for use in promoting myelination and/or neuroprotection in a subject in need thereof.

In one embodiment, the inhibitor of VEGFR-1 according to the present invention promotes myelination and/or neuroprotection in a subject in need thereof, and thus may be used to treat or prevent a demyelinating disease in a subject in need thereof. Thus, the present invention also relates to an inhibitor of vascular endothelial growth factor receptor 1 (VEGFR-1) for use in treating or preventing a demyelinating disease in a subject in need thereof.

In one embodiment, the inhibitor of VEGFR-1 according to the present invention is a natural compound, i.e. a compound that can be found in nature. In one embodiment, the inhibitor of VEGFR-1 according to the present invention is a synthetic compound.

In one embodiment, the inhibitor of VEGFR-1 according to the present invention directly binds to VEGFR-1. Thus, in one embodiment, the inhibitor of VEGFR-1 according to the present invention is a compound able to inhibit VEGFR-1 function by binding directly to this receptor.

In one embodiment, the term “inhibit VEGFR-1 function” refers to a decreased function as compared to a reference function induced by VEGFR-1 activation, such as, for example, a function inferior or equal to 90%, 80%, 70%, 60%, 50%, 40%, 30%, 20%, 10%, or less of the reference function. In one embodiment, the inhibition of VEGFR-1 function is measured after contacting a cell with a compound tested for its impact on function, and the reference function correspond to a function induced by VEGFR-1 activation measured in a cell not contacted with said compound.

In one embodiment, the inhibitor of VEGFR-1 according to the present invention is selected from the group comprising or consisting of small organic molecules, peptides, antibodies, antibody fragments and antibody mimetics. In one embodiment, the inhibitor of VEGFR-1 is a small organic molecule, a peptide or an antibody or fragment thereof.

In one embodiment, the inhibitor of VEGFR-1 according to the present invention is an antibody or an antibody fragment thereof directed against VEGFR-1.

Said antibody or fragment thereof may, for example, impair the binding of a ligand to VEGFR-1. Thus, in one embodiment, the antibody or antibody fragment thereof directed against VEGFR-1 is a blocking antibody or fragment thereof.

Examples of antibodies or antibody fragments binding to VEGFR-1 include, without limitation, antibodies or fragments thereof produced by the following hybridomas: KM1730 (deposited as FERM BP-5697), KM1731 (deposited as FERM BP-5718), KM1732 (deposited as FERM BP-5698), KM1748 (deposited as FERM BP-5699), KM1750 (deposited as FERM BP-5700) disclosed in International Applications WO 98/22616 and WO 99/59636.

Examples of other antibodies or antibody fragments include IMC-18F (icrucumab), D16F7 and MF1 antibodies.

The heavy and light chains of icrucumab are provided in SEQ ID NO: 2 and SEQ ID NO: 3., respectively.

SEQ ID NO: 2
QAQVVESGGGVVQSGRSLRLSCAASGFAFSSYGMHWVRQAPGKGLEWVAV
IWYDGSNKYYADSVRGRFTISRDNSENTLYLQMNSLRAEDTAVYYCARDH
YGSGVHHYFYYGLDVWGQGTTVTVSSASTKGPSVFPLAPSSKSTSGGTAA
LGCLVKDYFPEPVTVSWNSGALTSGVHTFPAVLQSSGLYSLSSVVTVPSS
SLGTQTYICNVNHKPSNTKVDKRVEPKSCDKTHTCPPCPAPELLGGPSVF
LFPPKPKDTLMISRTPEVTCVVVDVSHEDPEVKFNWYVDGVEVHNAKTKP
REEQYNSTYRVVSVLTVLHQDWLNGKEYKCKVSNKALPAPIEKTISKAKG
QPREPQVYTLPPSREEMTKNQVSLTCLVKGFYPSDIAVEWESNGQPENNY
KTTPPVLDSDGSFFLYSKLTVDKSRWQQGNVFSCSVMHEALHNHYTQKSL
SLSPGK
SEQ ID NO: 3
EIVLTQSPGTLSLSPGERATLSCRASQSVSSSYLAWYQQKPGQAPRLLIY
GASSRATGIPDRFSGSGSGTDFTLTISRLEPEDFAVYYCQQYGSSPLTFG
GGTKVEIKRTVAAPSVFIFPPSDEQLKSGTASVVCLLNNFYPREAKVQWK
VDNALQSGNSQESVTEQDSKDSTYSLSSTLTLSKADYEKHKVYACEVTHQ
GLSSPVTKSFNRGEC

The heavy and light chain variables regions of D16F7 are provided in SEQ ID NO: 4 and SEQ ID NO: 5, respectively.

SEQ ID NO: 4
MGWSSIILFLVATASGVHSQYQQQVGSVWKPGASYKLSCKASGYWTNYWM
HWYKQRPGQGLEWIGEIYPRNGNTNYDEKFKGKATLTIDTSSSTAYMQLS
SLTSEDSAVYYCATYLGDYWGQGTTLTVSS
SEQ ID NO: 5
MKLPVRLLVLMFWIPASSSDVLMTQTPLSLPVSLGDQASISCRSSQSIVH
SNGNTYLEWFLQKPGQSPKLLIYKVSNRFSGIPDRFSGSGSGTDFTLKIS
RVEAEDLGVYFCFQGSHVPYTFGGGTKLEIK

Antibodies directed against VEGFR-1 can be obtained according to known methods by administering the appropriate antigen or epitope to a host animal selected, e.g., from pigs, cows, horses, rabbits, goats, sheep, and mice, among others. Various adjuvants known in the art can be used to enhance antibody production. Although antibodies useful in practicing the invention can be polyclonal, monoclonal antibodies are preferred. Monoclonal antibodies directed to VEGFR-1 can be prepared and isolated using any technique that provides for the production of antibody molecules by continuous cell lines in culture. Techniques for production and isolation include but, are not limited to, the hybridoma technique originally described by Kohler and Milstein (1975); the human B-cell hybridoma technique (Cote et al., 1983); and the EBV-hybridoma technique (Cole et al. 1985). Alternatively, techniques described for the production of single chain antibodies (see, e.g., U.S. Pat. No. 4,946,778) can be adapted to produce single chain antibodies directed to VEGFR-1.

Examples of antibody fragments include, but are not limited to, F(ab′)2 fragments, which can be generated by pepsin digestion of an intact antibody molecule, and Fab fragments, which can be generated by reducing the disulfide bridges of the F(ab′)2 fragments. Other examples of antibody fragments include, without limitation, Fv and in particular scFv, a Fd, a defucosylated antibody, a diabody, a triabody and a tetrabody, a unibody, a domain antibody, and a nanobody. Alternatively, Fab and/or scFv expression libraries can be constructed to allow rapid identification of fragments having the desired specificity to VEGFR-1.

Humanized antibodies (or fragment thereof) directed to VEGFR-1 can also be prepared according to known techniques. “Humanized antibodies” are forms of non-human (e.g., rodent) chimeric antibodies that contain minimal sequence derived from non-human immunoglobulin. For the most part, humanized antibodies are human immunoglobulins (recipient antibody) in which residues from a hypervariable region (CDRs) of the recipient are replaced by residues from a hypervariable region of a non-human species (donor antibody) such as mouse, rat, rabbit, or nonhuman primate having the desired specificity, affinity and capacity. In some instances, framework region (FR) residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FRs are those of a human immunoglobulin sequence. The humanized antibody optionally also will comprise at least a portion of an immunoglobulin constant region (Fc), typically that of a human immunoglobulin. Methods for making humanized antibodies are described, for example, by Winter (U.S. Pat. No. 5,225,539) and Boss (Celltech, U.S. Pat. No. 4,816,397).

In one embodiment, the inhibitor of VEGFR-1 according to the present invention is an antibody mimetic. As used herein, antibody mimetics are organic compounds that, like antibodies, can specifically bind antigens, but that are not structurally related to antibodies.

Examples of antibody mimetics include, without limitation, an affibody, an alphabody, an armadillo repeat protein-based scaffold, a knottin, a domain Kunitz peptide, an affilin, an affitin, an adnectin, an atrimer, an evasin, a DARPin, an anticalin, an avimer, a fynomer, a versabody or a duocalin.

In one embodiment, the inhibitor of VEGFR-1 according to the present invention is a small organic molecule.

In one embodiment, the small organic molecule has a size up to about 5000 g/mol, more preferably up to 2000 g/mol, and more preferably up to about 1000 g/mol.

In one embodiment, the inhibitor of VEGFR-1 according to the present invention is ZM306416 (CB 676475, CAS number 690206-97-4), or a salt or a derivative thereof. The formula of ZM306416 is provided herein below:

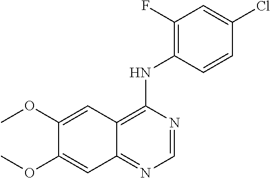 [SMILES: COc1cc2ncnc(Nc3ccc(Cl)cc3F)c2cc1OC]

ZM306416 is commercially available on supplier websites (https://www.selleckchem.com/products/zm-306416.html, https://www.tocris.com/products/zm-306416-hydrochloride_2499, https://www.abcam.com/zm306416-hydrochloride-vegfr12-tyrosine-kinase-activity-inhibitor-ab144576.html).

In one embodiment, the inhibitor of VEGFR-1 according to the present invention is SU14813 (CAS number 627908-92-3), or a salt or derivative thereof. The formula of SU14813 is provided hereinbelow:

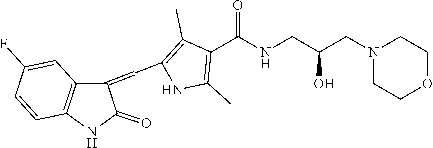 [SMILES: Cc1[nH]c(C=C2C(=O)Nc3ccc(F)cc32)c(C)c1C(=O)NC[C@H](O)CN1CCOCC1]

SU14813 is commercially available on supplier websites ((https://www.medchemexpress.com/SU14813.html, https://www.selleckchem.com/products/su14813.html, https://www.apexbt.com/su14813.html).

In one embodiment, the inhibitor of VEGFR-1 according to the present invention is a peptide.

Examples of peptides binding to VEGFR-1 are well known by the skilled artisan in the art, and include, without limitation, the anti VEGFR-1 peptide of sequence GNQWFI (SEQ ID NO: 6), the vasotide peptide of sequence _{D}(CLPRC) (SEQ ID NO: 7), the peptide F56 of sequence WHSDMEWWYLLG (SEQ ID NO: 8), the peptide A4 of sequence TEGRELVIPARVT (SEQ ID NO: 9) and the peptide B3 of sequence IPARVTS (SEQ ID NO: 10).

In one embodiment, the inhibitor of VEGFR-1 according to the present invention is a peptide selected from the group comprising or consisting of the peptides with SEQ ID NOs: 6 to 10 and combinations thereof. In one embodiment, the inhibitor of VEGFR-1 according to the present invention is the anti VEGFR-1 peptide of sequence GNQWFI (SEQ ID NO: 6).

In one embodiment, the inhibitor of VEGFR-1 according to the present invention is a nucleic acid, in particular a nucleic acid targeting the gene encoding for VEGFR-1, thereby inhibiting the expression of VEGFR-1.

In one embodiment, the inhibitor of VEGFR-1 according to the present invention is a nucleic acid, in particular a nucleic acid targeting the gene encoding for VEGFR-1 in oligodendrocyte lineage cells and/or Schwann cells, thereby inhibiting the expression of VEGFR-1 in oligodendrocyte lineage cells and/or Schwann cells.

In one embodiment, the term “inhibit the expression of VEGFR-1” refers to a decreased expression level as compared to a reference expression level of VEGFR-1, such as, for example, a level inferior or equal to 90%, 80%, 70%, 60%, 50%, 40%, 30%, 20%, 10%, or less of the reference expression level. In one embodiment, the inhibition of expression of VEGFR-1 is measured after contacting a cell with a compound tested for its impact on expression, and the reference expression level correspond to an expression level of VEGFR-1 measured in a cell not contacted with said compound.

Methods to determine the level of expression of genes are well-known to the skilled artisan, and include, without limitation, determining the transcriptome and/or the proteome.

In one embodiment, the level of expression is assessed at the transcription level (i.e., at the mRNA level).

In vitro methods for assessing the transcription level of a gene are well known in the prior art. Examples of such methods include, but are not limited to, RT-PCR, RT-qPCR, Northern Blot, hybridization techniques such as, for example, use of microarrays, and combination thereof including but not limited to, hybridization of amplicons obtained by RT-PCR, sequencing such as, for example, next-generation DNA sequencing (NGS) or RNA-seq (also known as “Whole Transcriptome Shotgun Sequencing”) and the like.

In one embodiment, the level of expression is assessed at the translation level (i.e., at the protein level).

In vitro methods for assessing the translation level of a gene are well-known in the art. Examples of such methods include, but are not limited to, immunohistochemistry, Multiplex methods (Luminex), western blot, enzyme-linked immunosorbent assay (ELISA), sandwich ELISA, fluorescent-linked immunosorbent assay (FLISA), enzyme immunoassay (EIA), radioimmunoassay (RIA), flow cytometry (FACS) and the like.

Examples of nucleic acids that may be used in the present invention include, without limitation, RNA interference molecules, oligonucleotide antisense (including, without limitation, antisense RNA or DNA molecules), ribozymes, aptamers and morpholinos.

As used herein, RNA interference (RNAi) is a gene-silencing process that targets mRNA hence lowering protein expression. Examples of RNAi substrates regulating this process include, without limitation, shRNAs (short hairpin or small hairpin RNA), siRNAs (small interfering RNA) and miRNAs (microRNA).

Thus, in one embodiment, the inhibitor of VEGFR-1 is an RNA interference molecule, preferably selected from the group comprising or consisting of shRNAs, siRNAs, miRNAs and combinations thereof.

shRNAs are RNAs that contains a loop structure that is processed to siRNA and also leads to the degradation of mRNAs in a sequence-specific manner dependent upon complementary binding of the target mRNA.

Examples of shRNAs targeting VEGFR-1 include, without limitation, the following shRNAs:

| (SEQ ID NO: 15) | |
|---|---|
| | 5′GGTCATAGAAGGAACCAATAAGACGGTTA′3, |
| (SEQ ID NO: 16) | |
| | 5′TCACTCTGAACCTTGTCATCAAGAATGTG′3, |
| (SEQ ID NO: 17) | |
| | 5′CTCGGTGACCTGCTTCAAGCCAATGTCCA′3, |
| (SEQ ID NO: 18) | |
| | 5′GAGCATCTATCAGGCAGCGGATTGACCAA′3. |

In one embodiment, the inhibitor of VEGFR-1 according to the present invention is a shRNA, preferably a shRNA selected from the group comprising or consisting of shRNAs with SEQ ID NOs: 15 to 18, and combinations thereof.

siRNAs are double stranded RNA, comprising an antisense (or guide) strand and a sense (or passenger) strand, which form a duplex 19 to 25 bp in length with 3′ dinucleotide overhangs. These siRNAs associate with helicase and nuclease molecules and form a large complex, termed RNA-induced silencing complex, which unwinds siRNA and directs sequence-specific degradation of mRNA.

Examples of siRNAs targeting VEGFR-1 include, without limitation, the following siRNAs:

| | sens: |
|---|---|
| (SEQ ID NO: 11) | |
| | 5′-GGCCAGCACAUAGGAGAGATT-3′; |
| | antisens: |
| (SEQ ID NO: 12) | |
| | 5′-UCUCUCCUAUGUGCUGGCCTT-3 |
| | sens: |
| (SEQ ID NO: 13) | |
| | 5′-BCU GAG UUU AAA AGG CAC CCT TB-3′; |
| | antisens: |
| (SEQ ID NO: 14) | |
| | 5′-GGG UGC CUU UUA AAC UCA GTST-3′, |

wherein “T” represents unpaired deoxythymidines, “S” represents one phosphorothioate linkage and “B” represents two inverted 2′-deoxy abasic nucleotides.

In one embodiment, the inhibitor of VEGFR-1 according to the present invention is a siRNA, preferably a siRNA selected from the group comprising or consisting of siRNA with SEQ ID NOs: 11 to 14, and combinations thereof.

miRNAs are gene-regulatory RNAs that are loaded onto the RNA-induced silencing complex (RISC) and interact with partially-complementary targets on mRNA to suppress protein expression. The miRNA is generally single-stranded, and on loading onto RISC, the miRNA “guide” sequence (also referred as the seed region) is held on the surface of RISC where it can interact with the target mRNA. The targets recognized by the miRNA guide sequence are most commonly on the 3′-untranslated region (UTR) of an RNA. Binding can suppress assembly of an initiation complex on the 5′ cap of an mRNA because the mRNA is bound into a circular shape at the initiation of translation, bringing the 3′-UTR and 5′-UTR close together.

shRNAs, siRNAs and miRNAs targeting VEGFR-1 are commercially available through the websites of products supplies.

In one embodiment, the inhibitor of VEGFR-1 according to the present invention is an anti-sense oligonucleotide. Anti-sense oligonucleotides, including anti-sense RNA molecules and anti-sense DNA molecules, would act to directly block the translation of VEGFR-1 mRNA by binding thereto and thus preventing protein translation or increasing mRNA degradation, thus decreasing the level of VEGFR-1, and thus activity, in a cell. For example, antisense oligonucleotides of at least about 15 bases and complementary to unique regions of the mRNA transcript sequence encoding VEGFR-1 can be synthesized, e.g., by conventional phosphodiester techniques and administered by e.g., intravenous injection or infusion. Methods for using antisense techniques for specifically inhibiting gene expression of genes whose sequence is known are well known in the art (e.g. see U.S. Pat. Nos. 6,566,135; 6,566,131; 6,365,354; 6,410,323; 6,107,091; 6,046,321; and 5,981,732, which are incorporated herein by reference).

In one embodiment, the inhibitor of VEGFR-1 according to the present invention is a ribozyme. Ribozymes are enzymatic RNA molecules capable of catalyzing the specific cleavage of RNA. The mechanism of ribozyme action involves sequence specific hybridization of the ribozyme molecule to complementary target RNA, followed by endonucleolytic cleavage. Engineered hairpin or hammerhead motif ribozyme molecules that specifically and efficiently catalyze endonucleolytic cleavage of VEGFR-1 mRNA sequences are thereby useful within the scope of the present invention. Specific ribozyme cleavage sites within any potential RNA target are initially identified by scanning the target molecule for ribozyme cleavage sites, which typically include the following sequences, GUA, GUU, and GUC. Once identified, short RNA sequences of between about 15 and 20 ribonucleotides corresponding to the region of the target gene containing the cleavage site can be evaluated for predicted structural features, such as secondary structure, that can render the oligonucleotide sequence unsuitable. The suitability of candidate targets can also be evaluated by testing their accessibility to hybridization with complementary oligonucleotides, using, e.g., ribonuclease protection assays.

In one embodiment, the inhibitor of VEGFR-1 according to the present invention is an aptamer. Aptamers are short, single-stranded DNA or RNA (ssDNA or ssRNA) molecules that can selectively bind to a specific target, including proteins, peptides, carbohydrates, small molecules, toxins, and even live cells. Aptamer binding is determined by its tertiary structure, and involves three-dimensional, shape-dependent interactions as well as hydrophobic interactions, base-stacking, and intercalation.

In one embodiment, the inhibitor of VEGFR-1 according to the present invention is a morpholino. Morpholinos are a type of oligomer molecule (colloquially, an oligo) used in molecular biology to modify gene expression. The molecular structure has a backbone of methylenemorpholine rings and phosphorodiamidate linkages. Morpholinos block access of other molecules to small (˜25 base) specific sequences of the base-pairing surfaces of ribonucleic acid (RNA).

In one embodiment, the inhibitor of VEGFR-1 according to the present invention is specific for VEGFR-1.

In one embodiment, the inhibitor of VEGFR-1 according to the present invention does not bind to proteins other than VEGFR-1. In one embodiment, the inhibitor of VEGFR-1 according to the present invention does not inhibit the expression of proteins other than VEGFR-1.

In one embodiment, the inhibitor of VEGFR-1 according to the present invention is selective for VEGRF-1. In one embodiment, the inhibitor of VEGFR-1 according to the present invention is selective for VEGFR-1 over VEGFR-2 and VEGFR-3.

In one embodiment, the inhibitor of VEGFR-1 according to the present invention does not bind VEGFR-2 and/or VEGFR-3, or binds VEGFR-2 and/or VEGFR-3 with lower affinity than VEGFR-1.

In one embodiment, the inhibitor of VEGFR-1 according to the present invention binds VEGFR-1 with an affinity at least 10-fold, 25-fold, 50-fold, 75-fold, 80-fold, 90-fold, 95 fold, 100-fold, 125-fold, 150-fold, 200-fold, 250-fold, 300-fold, 350-fold, 400-fold, 450-fold, or 500-fold higher than the affinity for the other VEGFR, in particular VEGFR-2 and VEGFR-3.

Affinity can be relatively determined by binding or competitive binding assays, using, e.g., Biacore instruments.

In one embodiment, the inhibitor of VEGFR-1 according to the present invention does not inhibit VEGFR-2 and/or VEGFR-3 function(s), or inhibit VEGFR-2 and/or VEGFR-3 function(s) to a lower extent than VEGFR-1 function.

In one embodiment, the inhibitor of VEGFR-1 according to the present invention inhibits VEGFR-1 function 1.5 times, 2 times, 2.5 times, 5 times, 10 times, 50 times, 100 times, 500 times, or 1000 times higher than VEGFR-2 and/or VEGFR-3 function.

In one embodiment, the inhibitor of VEGFR-1 according to the present invention does not inhibit VEGFR-2 and/or VEGFR-3 expression or inhibit VEGFR-2 and/or VEGFR-3 expression to a lower extent than VEGFR-1 expression.

In one embodiment, the inhibitor of VEGFR-1 according to the present invention inhibits VEGFR-1 expression 1.5 times, 2 times, 2.5 times, 5 times, 10 times, 50 times, 100 times, 500 times, or 1000 times higher than VEGFR-2 and/or VEGFR-3 expression.

In one embodiment, the inhibitor of VEGFR-1 according to the present invention is the only one therapeutic agent for use in promoting myelination and neuroprotection or for use in treating or preventing a demyelinating disease in a subject in need thereof.

Thus, in one embodiment, the inhibitor of VEGFR-1 is to be used as a monotherapy.

In one embodiment, the inhibitor of VEGFR-1 according to the present invention is to be administered in combination with another therapeutic agent.

In one embodiment, the other therapeutic agent is an anti-inflammatory drug.

In one embodiment, the anti-inflammatory drug is an immunomodulator or an immunosuppressant.

Examples of anti-inflammatory drugs include, without limitation: interferon beta, Glatiramer acetate (CAS 147245-92-9); Fingolimod (CAS 162359-55-9), Dimethyl fumarate (CAS 624-49-7), Diroximel fumarate (CAS 1577222-14-0), Teriflunomide (CAS 163451-81-8), Siponimod (CAS 1230487-00-9), Cladribine (CAS 4291-63-8), Natalizumab (CAS 189261-10-7), Ocrelizumab (CAS 637334-45-3), Alemtuzumab (CAS 216503-57-0), Cyclophosphamide (CAS 50-18-0), Azathioprine (CAS 446-86-6) and Mitoxandrone (CAS 65271-80-9).

The formulas of some anti-inflammatory drugs are provided hereinbelow:

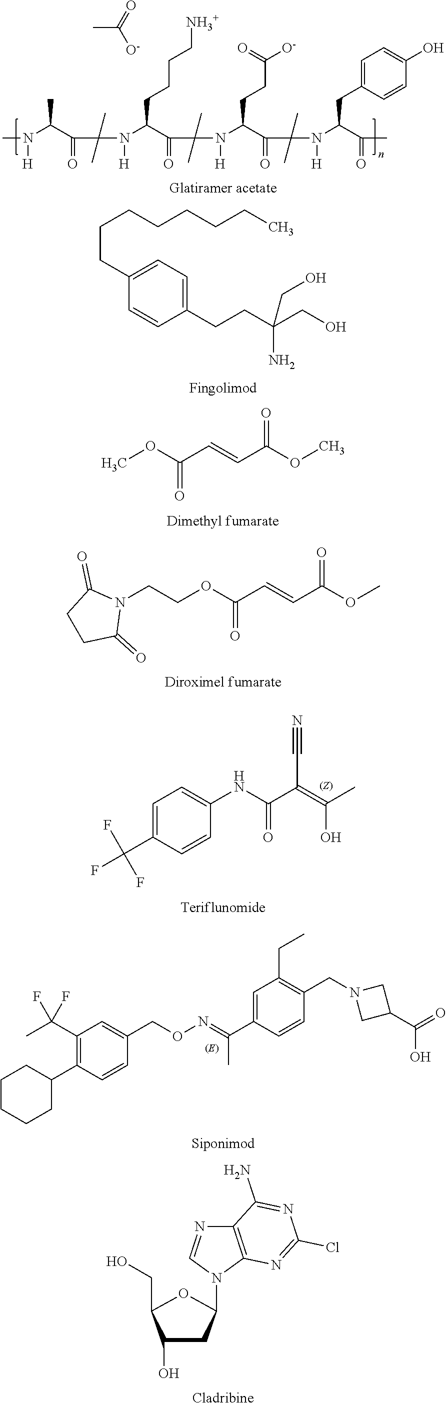

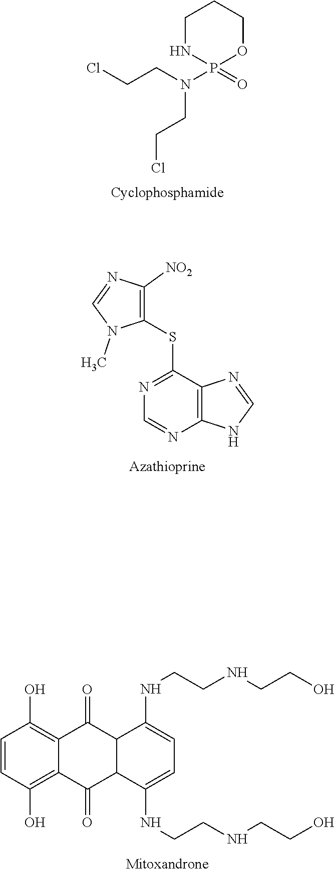 [SMILES: O=P1(N(CCCl)CCCl)NCCCO1 ; Cn1cnc([N+](=O)[O-])c1Sc1ncnc2[nH]cnc12 ; O=C1c2c(O)ccc(O)c2C(=O)C2C(NCCNCCO)=CC=C(NCCNCCO)C12]

Thus, in one embodiment, the inhibitor of VEGFR-1 according to the present invention is to be administered in combination with an anti-inflammatory drug selected from the group comprising or consisting of interferon beta, Glatiramer acetate, Fingolimod, Dimethyl fumarate, Diroximel fumarate, Teriflunomide, Siponimod, Cladribine, Natalizumab, Ocrelizumab, Alemtuzumab, Cyclophosphamide, Azathioprine Mitoxandrone and combinations thereof.

The present invention further relates to a kit of parts comprising, in one part, at least one inhibitor of VEGFR-1 and, in a second part, at least one anti-inflammatory drug as described hereinabove.

In one embodiment, the kit of parts as described hereinabove is for use in promoting myelination and/or neuroprotection in a subject in need thereof.

In one embodiment, the kit of parts as described hereinabove is for use in treating or preventing a demyelinating disease in a subject in need thereof.

In one embodiment, the at least one inhibitor of VEGFR-1 and the at least one inflammatory drug are to be administered simultaneously, separately or sequentially.

In one embodiment, the at least one inhibitor of VEGFR-1 and the at least one inflammatory drug are to be administered by the same routes of administration. In one embodiment, the at least one inhibitor of VEGFR-1 and the at least one inflammatory drug are to be administered by different routes of administration.

In one embodiment, the subject is affected, preferably diagnosed, with a demyelinating disease.

In one embodiment, the demyelinating disease affects the central nervous system (CNS). Thus, in one embodiment, the subject is affected, preferably diagnosed, with a demyelinating disease of the central nervous system (CNS).

As used herein, demyelinating diseases of the CNS can be classified according to their pathogenesis into several categories: demyelination due to inflammatory processes, viral demyelination, demyelination caused by acquired metabolic dysfunctions, hypoxic-ischaemic forms of demyelination and demyelination caused by focal compression.

Symptoms of demyelinating diseases include, but are not limited to, vision loss, muscle weakness, muscle stiffness, muscle spasms, bladder and bowels problems and sensory changes.

Examples of demyelinating diseases of the CNS include, without limitation, multiple sclerosis, neuromyelitis optica or Devic's disease, acute disseminated encephalomyelitis (ADEM), optic neuritis, cerebral ischemia, leukodystrophies, traumatic brain injury (TBI) and transverse myelitis.

In one embodiment, the demyelinating disease is selected from the group comprising or consisting of multiple sclerosis, neuromyelitis optica or Devic's disease, acute disseminated encephalomyelitis (ADEM), optic neuritis, cerebral ischemia, leukodystrophies, traumatic brain injury (TBI) and transverse myelitis.

In one embodiment, the demyelinating disease is selected from the group comprising or consisting of multiple sclerosis, optic neuritis, cerebral ischemia, leukodystrophies and traumatic brain injury (TBI).

In one embodiment, the subject is not affected with multiple sclerosis.

In one embodiment, the subject is affected, preferably diagnosed, with multiple sclerosis.

Symptoms of multiple sclerosis are diverse and include, without limitation, fatigue, vision problems, numbness and tingling, muscle spasms, stiffness and weakness, mobility problems, pain, problems with thinking, learning and planning, depression and anxiety, sexual problems, bladder problems, bowel problems and speech and swallowing difficulties.

Tests for diagnosing a demyelinating disease, such as a multiple sclerosis, are well-known by the skilled artisan in the art and include, without limitation, MRI scans of the brain and spinal cord, spinal fluid analysis (lumbar puncture), and evoked potential tests.

It is well known by the skilled artisan in the art that multiple classifications of MS subtypes exist in the literature. According to the National MS Society, several forms of multiple sclerosis can be encountered: clinically isolated syndrome (CIS), relapsing remitting multiple sclerosis (RRMS), secondary progressive multiple sclerosis (SPMS) and primary progressive multiple sclerosis (PPMS). The National MS Society gives definition of these multiple sclerosis disease subtypes that are provided herein below.

In one embodiment, the multiple sclerosis is selected from the group consisting of clinically isolated syndrome (CIS), relapsing remitting multiple sclerosis (RRMS), primary progressive multiple sclerosis (PPMS), and secondary progressive multiple sclerosis (SPMS).

In one embodiment, the multiple sclerosis is a progressive form of the disease, such as, for example, PPMS and SPMS.

In one embodiment, the multiple sclerosis is CIS. As used herein, CIS is a first episode of neurologic symptoms caused by inflammation and demyelination in the central nervous system. The episode, which by definition must last for at least 24 hours, is characteristic of multiple sclerosis but does not yet meet the criteria for a diagnosis of multiple sclerosis. However, it is possible to diagnose multiple sclerosis in a person with CIS who also has specific findings on brain MRI that provide evidence of an earlier episode of damage in a different location and indicate active inflammation in a region other than the one causing the current symptoms.

In one embodiment, the multiple sclerosis is RRMS. As used herein, RRMS is characterized by clearly defined attacks of new or increasing neurologic symptoms. These attacks, also called relapses or exacerbations, are followed by periods of partial or complete recovery (remissions). During remissions, all symptoms may disappear, or some symptoms may continue and become permanent. However, there is no apparent progression of the disease during the periods of remission. RRMS can be further characterized as either active (with relapses and/or evidence of new MRI activity over a specified period of time) or not active, as well as worsening (a confirmed increase in disability following a relapse) or not worsening.

In one embodiment, the multiple sclerosis is progressive multiple sclerosis.

In one embodiment, the multiple sclerosis is PPMS. As used herein, PPMS is characterized by worsening neurologic function (accumulation of disability) from the onset of symptoms, without early relapses or remissions. PPMS can be further characterized as either active (with an occasional relapse and/or evidence of new MRI activity over a specified period of time) or not active, as well as with progression (evidence of disability accumulation over time, with or without relapse or new MRI activity) or without progression.

In one embodiment, the multiple sclerosis is SPMS. As used herein, SPMS follows an initial relapsing-remitting course. Some people who are diagnosed with RRMS will eventually transition to a secondary progressive course in which there is a progressive worsening of neurologic function (accumulation of disability) over time. SPMS can be further characterized as either active (with relapses and/or evidence of new MRI activity during a specified period of time) or not active, as well as with progression (evidence of disability accumulation over time, with or without relapses or new MRI activity) or without progression.

In one embodiment, the demyelinating disease is optic neuritis. As used herein, the optic neuritis is an inflammation of the optic nerve. Clinical features include retro-orbital pain that is aggravated by eye movement, loss of color vision, and contrast sensitivity that may progress to severe visual loss, an afferent pupillary defect (Marcus-Gunn pupil), and in some instances, optic disc hyperemia and swelling. Inflammation may occur in the portion of the nerve within the globe (neuropapillitis or anterior optic neuritis) or the portion behind the globe (retrobulbar neuritis or posterior optic neuritis).

In one embodiment, the demyelinating disease is cerebral ischemia. As used herein, cerebral ischemia, also known as brain ischemia or cerebrovascular ischemia is an acute brain injury resulting from an insufficient amount of blood flow to the brain.

In one embodiment, the demyelinating disease is a leukodystrophy. As used herein, leukodystrophies are a group of rare, progressive, metabolic, genetic diseases that affect the brain, spinal cord and often the peripheral nerves. Each type of leukodystrophy is caused by a specific gene abnormality that leads to abnormal development or destruction of the white matter (myelin sheath) of the CNS.

In one embodiment, the demyelinating disease is a traumatic brain injury (TBI). As used herein, TBI is a form of acquired brain injury, occurring when a sudden trauma causes damage to the brain. TBI can result when the head suddenly and violently hits an object, or when an object pierces the skull and enters brain tissue.

In one embodiment, the demyelinating disease affects the peripheral nervous system (PNS). Thus, in one embodiment, the subject is affected, preferably diagnosed, with a demyelinating disease of the peripheral nervous system (PNS).

Examples of demyelinating diseases of the PNS include, without limitation, Guillain-Barré syndrome, chronic inflammatory demyelinating polyneuropathy, anti-MAG peripheral neuropathy, Charcot-Marie-Tooth diseases, Copper deficiency-associated conditions (peripheral neuropathy, myelopathy, and rarely optic neuropathy) and progressive inflammatory neuropathy.

In one embodiment, the subject is at risk of developing a demyelinating disease, and in particular, the subject is at risk of developing multiple sclerosis.

Examples of risk factors for developing multiple sclerosis, include, without limitation, age (between 20-40 years old), sex (woman), genetic factors, smoking, infections including exposure to viruses such as Epstein-Barr virus (EBV), mononucleosis, human herpes virus type 6 (HHV6) or mycoplasma pneumonia, Vitamin D deficiency, Vitamin B12 deficiency.

In one embodiment, the subject has received, is receiving or will receive a treatment for demyelination. In one embodiment, the subject has received, is receiving or will receive a treatment for treating the demyelinating disease as described hereinabove.

In one embodiment, the subject has received, is receiving or will receive a treatment for treating multiple sclerosis.

Examples of treatments for multiple sclerosis, include, without limitation, physical therapy, anti-inflammatory drugs and muscle-relaxing drugs.

In one embodiment, the subject has received, is receiving or will receive an anti-inflammatory drug. Examples of anti-inflammatory drugs are provided hereinabove.

In one embodiment, the subject has received, is receiving or will receive an immunomodulator or an immunosuppressant.

The present invention further relates to a composition comprising, consisting essentially of or consisting of an inhibitor of VEGFR-1 as described hereinabove, preferably wherein said composition is for use in promoting myelination and/or neuroprotection in a subject in need thereof.

The present invention further relates to a composition comprising, consisting essentially of or consisting of an inhibitor of VEGFR-1 as described hereinabove, preferably wherein said composition is for use in treating or preventing a demyelinating disease in a subject in need thereof.

As used herein, “consisting essentially of”, with reference to a composition, means that the inhibitor of VEGFR-1 is the only one therapeutic agent or agent with a biologic activity within said composition.

In one embodiment, the composition further comprises an anti-inflammatory drug as described hereinabove.

The present invention further relates to a pharmaceutical composition comprising the inhibitor of VEGFR-1 as described hereinabove and a pharmaceutically acceptable excipient, preferably wherein said pharmaceutical composition is for use in promoting myelination and/or neuroprotection in a subject in need thereof.

The present invention further relates to a pharmaceutical composition comprising the inhibitor of VEGFR-1 as described hereinabove and a pharmaceutically acceptable excipient, preferably wherein said pharmaceutical composition is for use in treating or preventing a demyelinating disease in a subject in need thereof.

In one embodiment, the pharmaceutical composition further comprises an anti-inflammatory drug as described hereinabove.

Within the meaning of the invention, the expression “pharmaceutical composition” refers to a composition comprising an active principle in association with a pharmaceutically acceptable vehicle or excipient. A pharmaceutical composition is for therapeutic use, and relates to health.

The term “pharmaceutically acceptable excipient” includes any and all solvents, dispersion media, coatings, antibacterial and antifungal agents, isotonic and absorption delaying agents and the like. Said excipient does not produce an adverse, allergic or other untoward reaction when administered to an animal, preferably a human. For human administration, preparations should meet sterility, pyrogenicity, and general safety and purity standards as required by regulatory offices, such as, for example, FDA Office or EMA.

Pharmaceutically acceptable excipients that may be used in these compositions include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances (for example sodium carboxymethylcellulose), polyethylene glycol, polyacrylates, waxes, polyethylene-polyoxypropylene-block polymers, polyethylene glycol and wool fat.

The present invention further relates to a medicament comprising, consisting essentially of or consisting of an inhibitor of VEGFR-1 as described hereinabove, preferably wherein said medicament is for use in promoting myelination and/or neuroprotection in a subject in need thereof.

The present invention further relates to a medicament comprising, consisting essentially of or consisting of an inhibitor of VEGFR-1 as described hereinabove, preferably wherein said medicament is for use in treating or preventing a demyelinating disease in a subject in need thereof.

In one embodiment, the medicament further comprises an anti-inflammatory drug as described hereinabove.

The present invention further relates to the use of an inhibitor of VEGFR-1 as described hereinabove in the manufacture of a medicament for promoting myelination and/or neuroprotection, or for treating or preventing a demyelinating disease, such as multiple sclerosis, in a subject in need thereof.

The inhibitor of VEGFR-1, the components of the kit, the combination, the composition, the pharmaceutical composition or the medicament according to the present invention is/are formulated for administration to the subject.

The inhibitor of VEGFR-1, the components of the kit, the combination, the composition, the pharmaceutical composition or the medicament may be administered orally, by injection, topically, nasally, by inhalation, buccally, rectally, intratracheally, by endoscopy, transmucosally, or by percutaneous administration. The term injection used herein includes subcutaneous, intravenous (IV), intramuscular, intra-articular, intra-synovial, intrasternal, intrathecal, intrahepatic, intralesional, perispinal and intracranial injection or infusion techniques.

In one embodiment, the inhibitor of VEGFR-1, the components of the kit, the combination, the composition, the pharmaceutical composition or the medicament is to be administered orally, nasally or by injection.

Examples of forms adapted for injection include, but are not limited to, solutions, such as, for example, sterile aqueous solutions, gels, dispersions, emulsions, suspensions, solid forms suitable for using to prepare solutions or suspensions upon the addition of a liquid prior to use, such as, for example, powder, liposomal forms and the like.

Examples of forms suitable for oral administration include, but are not limited to, tablets (including sustained-release tablets), hard capsules, powders, pills (including sugar-coated pills), capsules (including soft gelatin capsules), oral suspensions, oral solutions, and other similar forms.

Examples of forms suitable for nasal administration include, but are not limited to, sprays, nasal drops, nasal ointment and nasal spray solutions.

In one embodiment, the inhibitor of VEGFR-1, the components of the kit, the combination, the composition, the pharmaceutical composition or the medicament according to the present invention is/are to be administered in a therapeutically effective amount.

In one embodiment, the dose of the inhibitor of VEGFR-1 (preferably comprised in a kit, a combination, a composition, a pharmaceutical composition or a medicament according to the present invention) to be administered ranges from about 0.1 to about 1000 mg/kg, preferably from about 0.5 to about 100 mg/kg, more preferably from about 1 to about 50 mg/kg.

It will be understood that the dose of the inhibitor of VEGFR-1, the components of the kit or the combination according to the present invention will be decided by the attending physician within the scope of sound medical judgment. The specific dose for any particular subject will depend upon a variety of factors including the symptom being treated and the severity of the symptom; activity of the specific compounds employed; the specific composition employed, the age, body weight, general health, sex and diet of the patient; the time of administration, route of administration, and rate of excretion of the specific compounds employed; the duration of the treatment; drugs used in combination or coincidental with the specific compounds employed; and like factors well known in the medical arts. For example, it is well known within the skill of the art to start doses of the compounds at levels lower than those required to achieve the desired therapeutic effect and to gradually increase the dosage until the desired effect is achieved.

In one embodiment, the inhibitor of VEGFR-1, the components of the kit, the combination, the composition, the pharmaceutical composition or the medicament according to the present invention is/are to be administered once.

In one embodiment, the inhibitor of VEGFR-1, the components of the kit, the combination, the composition, the pharmaceutical composition or the medicament of the present invention is/are to be administered several times.

In one embodiment, the inhibitor of VEGFR-1, the components of the kit, the combination, the composition, the pharmaceutical composition or the medicament according to the present invention is/are to be administered once a day (i.e. daily), every three days, every four days, every five days, every six days, every seven days, every eight days, every nine days, every ten days, every eleven days, every twelve days, every thirteen days, every fourteen days, every fifteen days, once a month, twice a month, once a week, twice a week, at least once a day, twice, or three times a day over a period determined by the skilled man in the art such as, for example, for at least a week, at least a month, for at least two months, at least a year, or more as needed for the rest of the subject's life.

In one embodiment, the inhibitor of VEGFR-1, the components of the kit, the combination, the composition, the pharmaceutical composition or the medicament according to the present invention is/are to be administered until complete healing of the subject.

In one embodiment, the inhibitor of VEGFR-1, the combination, the kit, the composition, the pharmaceutical composition, or the medicament according to the present invention promotes the myelination or the myelin regeneration of nerve fibers in a subject in need thereof. In one embodiment, the inhibitor of VEGFR-1, combination, kit, composition, pharmaceutical composition, or medicament promotes the survival, differentiation and/or maturation of the oligodendrocyte lineage cells and/or Schwann cells.

Thus, the present invention further relates a method for promoting survival, differentiation and/or maturation of the oligodendrocyte lineage cells and/or Schwann cells in a subject in need thereof, comprising administering to the subject an inhibitor of VEGFR-1, a combination, a kit, a composition, a pharmaceutical composition, or a medicament as described hereinabove. The present invention further relates to an inhibitor of VEGFR-1, a combination, a kit, a composition, a pharmaceutical composition, or a medicament as described hereinabove for use in promoting survival, differentiation and/or maturation of the oligodendrocyte lineage cells and/or Schwann cells in a subject in need thereof.

In one embodiment, an inhibitor of VEGFR-1, a combination, a kit, a composition, a pharmaceutical composition, or a medicament as described hereinabove prevents demyelination and/or neuronal loss in a subject.

Thus, the present invention further relates to a method for preventing demyelination and/or neuronal loss in a subject in need thereof, comprising administering to the subject an inhibitor of VEGFR-1, a combination, a kit, a composition, a pharmaceutical composition, or a medicament as described hereinabove. The present invention further relates to an inhibitor of VEGFR-1, a combination, a kit, a composition, a pharmaceutical composition, or a medicament as described hereinabove for use in preventing demyelination and/or neuronal loss in a subject in need thereof.

The present invention further relates to a method for treating or preventing a demyelinating disease, such as multiple sclerosis, in a subject in need thereof, comprising administering to the subject an inhibitor of VEGFR-1, a combination, a kit, a composition, a pharmaceutical composition, or a medicament as described hereinabove.

The present invention further relates to in vitro or ex-vivo methods for preventing demyelination or promoting remyelination of neurons, comprising contacting said neurons with an inhibitor of VEGFR-1 as described hereinabove.

The present invention further relates to in vitro or ex-vivo methods for preventing neuronal loss or promoting neuroprotection, comprising contacting said neurons with an inhibitor of VEGFR-1 as described hereinabove.

It will be understood by the skilled artisan in the art that an inhibitor of VEGFR-1 according to the present invention exhibits several of those properties, such as, for example, promoting remyelination and/or neuroprotection. By acting on myelin regeneration and/or neuronal protection, the inhibitor of VEGFR-1 according to the present invention may be used in remyelination therapies.

In one embodiment, said subject presents with signs of remyelination after administration of the inhibitor of VEGFR-1, the components of the kit, the combination, the composition, the pharmaceutical composition or the medicament according to the present invention.

In one embodiment, said subject presents with signs of reduction of demyelination after administration of the inhibitor of VEGFR-1, the components of the kit, the combination, the composition, the pharmaceutical composition or the medicament according to the present invention.

Means for assessing the level of myelination are well known by the skilled artisan in the art and include, for example, MRI and/or visual evoked potential analyses.

In one embodiment, said subject presents a reduction in at least one symptom of the demyelinating disease after administration of the inhibitor of VEGFR-1, the components of the kit, the combination, the composition, the pharmaceutical composition or the medicament according to the present invention.

In one embodiment, said subject presents a reduction of neuronal loss after administration of the inhibitor of VEGFR-1, the components of the kit, the combination, the composition, the pharmaceutical composition or the medicament according to the present invention.

In one embodiment, said subject has an improved lifespan and/or health span after administration of the inhibitor of VEGFR-1, the components of the kit, the combination, the composition, the pharmaceutical composition or the medicament according to the present invention.

### BRIEF DESCRIPTION OF THE DRAWINGS

FIG. 1 is a combination of fluorescence microscopy images showing that VEGFR-1 inhibitors promote neuroprotection of Purkinje cells in mouse organotypic cerebellar slices. Cerebellar slices, prepared from postnatal day 0 (P0), were cultured 10 days in vitro in (A) basal medium, or in the basal medium supplemented either with: (B) the pan-VEGFRs inhibitors (Lucitanib and MGCD-265), (C) the VEGFR-2/3 specific inhibitor (Telatinib), or (D) the VEGFR-1 selective and specific inhibitors (SU14813, ZM306416), respectively. All compounds were used at 500 nM, except ZM306416 that has been used at 30 μM. Purkinje cells are immunostained for Calbindin (CaBP, gray).

FIG. 2 is a combination of fluorescence microscopy images and a graph showing that VEGFR-1 inhibitors promote efficiently OPC differentiation in vitro. Rat primary OPC cultures in differentiation medium alone (A, basal), basal medium with 9 cis-RA (used as positive control, (B)), the pan-VEGFRs inhibitors Lucitanib and MGCD-265 (C), VEGFR-2/3 selective inhibitor Telatinib (D) or the selective VEGFR-1 inhibitor SU14813 (E). All compounds were tested at 500 nM. (F) Graph of the fold increase of MBP+Sox10+ oligodendrocytes relative to the basal condition, after 5 days in vitro. Data represent mean±SEM of N=3-4 experiments. Student t tests: *p<0.05, **p<0.01, ***p<0.001 relative to the basal condition. ≠p<0.05 for the t test comparison of VEGFR-2/3 inhibitor Telatinib versus VEGFR-1 selective inhibitor SU14813.

FIG. 3 is a combination of fluorescence microscopy images and a graph showing the effects on myelination in mouse cerebellar slices of pan-VEGFRs, VEGFR-2/3 and VEGFR-1 specific inhibitors. Cerebellar slices were treated with (A) basal medium alone, (B) 9 cis-retinoic acid (9-cis-RA, used as a positive control), (C) pan-VEGFRs inhibitor (Lucitanib), (D) VEGFR-2/3 selective inhibitor Telatinib or (E) VEGFR-1 specific inhibitor (ZM306416). MBP for myelin (white) and Calbindin (CaBP) for Purkinje cells (gray). (F) Graph of the myelination index after treatments with the pan-VEGFRs inhibitors (Lucitanib and MGCD-265), the VEGFR-2/3 inhibitor (Telatinib) and the VEGFR-1 selective and specific inhibitors (SU14813 and ZM306416), relative to the basal condition. Student t tests: ***p<0.001, ****p<0.0001 relative to the basal condition. ≠p<0.05 for the t test comparisons of VEGFR-2/3 inhibitor Telatinib versus VEGFR-1 selective inhibitors. ≠≠p<0.01, ≠≠p<0.001 for the t test comparisons of pan-VEGFR inhibitors versus VEGFR-1 selective inhibitors. VEGFR-1 inhibitors promote more efficiently myelination as compared to pan-VEGFRs and VEGFR-2/3 antagonists.

FIG. 4 is a combination of Western blots and graphs showing the detection of VEGFR-1 in rat primary OPC cultures. VEGFR-1 expression at the protein level decreases during oligodendrocyte differentiation in vitro. FIG. 4A: Western blot analysis of VEGFR-1 protein expression in rat primary OPC cultures in proliferation, 3 days and 5 of differentiation (experiments were done in triplicates). Western-blot were probed with an anti-VEGFR-1 antibody, an anti-MBP antibody and anti-GAPDH antibody (used as a loading control). FIG. 4B: Quantification of the Western blot by ImageLab BioRad. Levels of VEGFR-1 were normalized to GAPDH and presented as the relative expression levels to the proliferation condition. FIG. 4C: Levels of MBP normalized to GAPDH loading control. Data represent mean±SEM. N=2 to 5. Student t′test: *p≤0.05; **p≤0.01.

FIG. 5 is a combination of fluorescence microscopy images and graphs showing that shRNA knock-down of VEGFR-1 promotes oligodendrocyte differentiation. FIG. 5A: Lentiviral transduction of rat primary OPC cultures with VEGFR-1 shRNA, leads to 2-fold decrease of VEGFR-1 mRNA expression in rat primary OPC cultures, with respect to scrambled shRNA control cultures. FIG. 5B: MBP immunostaining of control and VEGFR-1 shRNA transduced rat primary OPC cultures, in basal medium at 5 days of differentiation. VEGFR1-knock-down favors OPC differentiation into MBP+ mature oligodendrocytes. FIG. 5C: MBP labeling of rat primary OPC cultures in basal medium alone or in basal medium supplemented with VEGFR-1 blocking peptide. Pharmacological inhibition of VEGFR-1 with a selective blocking peptide promotes oligodendrocyte differentiation. FIG. 5D: Fold increase of MBP+Olig2+ oligodendrocytes either after transduction with control or VEGFR-1-specific shRNA, or with VEGFR-1 specific blocking peptide. Data represent mean±SEM. N=1 to 2. Scale bar: 100 μm.

FIG. 6 is a combination of fluorescence microscopy images showing that VEGFR-1 blocking peptide promotes myelination in organotypic cerebellar slices. CaBP (grey) and MBP (white) immunostaining of mouse organotypic cerebellar slices, treated with basal condition or VEGFR-1-blocking peptide from day 5 to day 10 of cultures. Treatments with VEGFR1 blocking peptide enhances the density of MBP+CaBP+ myelinated axons.

### EXAMPLES

The present invention is further illustrated by the following examples.

### Materials and Methods

### Rat Primary OPC Cultures and Immunocytochemistry

Rat primary OPC cultures were obtained from neonatal pups (P0-P1). Brains were dissected, cerebellar cortices and meninges were removed. Brain tissues were next mechanically and enzymatically dissociated. After filtration and centrifugation, mixed cells of astrocytes, microglia and OPCs was cultured in DMEM medium on polyornithine-coated flasks. After 13-14 days of culture, microglial cells were removed by shaking during 2 h. OPCs were next collected after 18 h of shaking, followed by differential adhesion. OPCs purification was validated by immunostaining of Olig2 and Sox10. To induce differentiation, cells were kept in proliferation medium (DMEM-F12, 1% PS, FGF, PDGF, 1% laminin, Biotin, 2% B27, N1 mix) for 24 h and then switched to a differentiation medium with the same composition but deprived of FGF and PDGF. Cells were incubated at 37° C. (5% CO2) for 5 days and the medium was partially replaced every 2 days. Cells were then fixed in 2% paraformaldehyde and immunostained with Sox10 and MBP. Cells were incubated for 2 hours with primary antibodies Sox10 (Goat 1/100) and MBP (Rabbit 1/500) diluted in 4% BSA in PBS 0.1M/0.1% Triton. After washes, cells were then incubated with the secondary antibody (anti-Goat Alexa647, anti-Rabbit Alexa488 and Dapi) for 1 hour and finally washed in PBS 0.1M and mounted in Fluoromount medium. Flurorescence imaging were performed with a confocal microscopy NIKON A1R-HD25.

### Organotypic Slices and Immunohistochemistry

Organotypic cerebellar slices were generated from brains of postnatal day 0 (P0) and P7 mouse pups. Briefly, cerebella were dissected and cut into 350 μm slices. Cerebellar slices from the vermis were collected and then cultured on Millicell cell culture inserts (Millipore) in 6-wellplates in a medium composed of 50% Basal Medium Eagle, 25% Hank solution (HBSS), 25% inactivated horse serum, 5 mg/ml high-glucose, 1 mM L-glutamine, 0.2% penicillin-streptomycin. Slices were incubated at 37° C. (5% CO2) for 10 days (slices from P0) or 3 days (slices from P7). The culture medium was replaced every 2-3 days. For immunohistochemistry, slices were fixed in 4% paraformaldehyde and immunostained for CaBP and MBP. Briefly, slices were incubated in 4% BSA/0.2% triton for 1 hour and next incubated overnight with the following primary antibodies: rabbit anti-CaBP (dilution: 1/10000) and rat anti-MBP (dilution: 1/250). After several washes in PBS 0.1M, slices were then incubated with the secondary antibodies (anti-rabbit alexaFluor555, anti-mouse alexaFluor488) and Dapi for 2 hours and finally washed in PBS 0.1M and mounted in Fluoromount medium. Flurorescence imaging were performed with a confocal microscopy NIKON A1R-HD25.

### Neuroprotection

The neuroprotective effects of the drugs were assessed by evaluating the density of Purkinje cells in basal medium alone and in basal medium supplemented with VEGFRs inhibitors. Organotypic cerebellar slices obtained from P0 mouse (as described above) were cultured for 10 days in vitro in basal medium alone, or in the basal medium supplemented either with pan-VEGFRs inhibitors, VEGFR-2/3 specific inhibitor (Telatinib) or with VEGFR-1 selective and specific inhibitors (SU14813 and ZM306416), respectively. All compounds were used at 500 nM, except ZM306416 that was used at 30 μM. After treatments, slices were immunostained for CaBP, as described above.

### Oligodendrocytes Differentiation and Myelination

Oligodendrocytes differentiation was assessed by analyzing the fold increase of MBP⁺Sox10⁺ cells in treated conditions relative to the basal condition. The percentage of MBP⁺Sox10⁺ cells were quantified after treatments with pan-VEGFRs and selective VEGFR-1 antagonists and compared with the basal medium. Results are shown as differentiation fold increase relative to the basal condition. Myelination was assessed using organotypic cerebellar slices generated from P7 mice cerebellum and the effect of VEGFRs sub-types antagonists on myelination was analyzed after immunostaining for Sox10 for oligodendroglia, MBP for myelin (white) and calbindin (gray). Slices were cultured in basal medium (control condition), or in the basal medium supplemented either with 9cis-RA (used as a positive control of myelination) or with VEGFR-1 selective and specific inhibitor (SU14813 and ZM306416), respectively. All compounds were used at 500 nM, except ZM306416 that has been used at 30 μM. The myelination index for each condition was quantified as the percentage of MBP+CaBP+ axons over the total CaBP+ axonal surface area and normalized relatively to basal condition.

### Western Blot

Cell lysis and denaturation. Rat primary OPC cultures coated onto B60 Petri dishes were lysed using RIPA Buffer (ThermoFisher Scientific), DTT (ThermoFisher Scientific) and Protease and Phosphatase Inhibitor (PPI, ThermoFisher Scientific), either after 48 hours in proliferation medium, or after 3 and 5 days in differentiation medium. The lysed cells were centrifuged for 10 min. at 13000 rpm at 4° C. The supernatant containing the proteins was recuperated and denatured using Laemmli 4X (BioRad) and β-mercaptoethanol before being heated for 5 min. at 95° C. The cell lysis was conserved at −80° C.

Gel electrophoresis and transfer. Equal amounts of protein for each sample were loaded into wells of a 4-20% TGX Gel (BioRad) and separated by SDS-PAGE at 100V for 1 h 30. The proteins were transferred to a PVDF membrane (BioRad) using the Trans-Blot Turbo. Ponceau stain on the membrane confirmed the transfer of proteins.

Protein detection. Membranes were blocked using the EveryBlot Blocking Buffer (BioRad) for 15-20 min. at room temperature, then incubated with the following primary antibodies diluted in blocking buffer overnight at 4° C. on a shaker: anti-VEGFR-1 (Abcam ab32152, 1:2000 dilution), anti-GAPDH (Cell Signaling CST2118, 1:2000 dilution) and anti-MBP (Genetex GTX133108, 1:5000 dilution). The next day, membranes were washed 4 times for 2 min. each with 1×TBS+0.1% Tween before incubation with horseradish peroxidase (HRP)-conjugated antibodies (Cell Signaling CST7074, 1:5000 dilution) for an hour at RT. Membranes were washed again 4 times for 2 min. with 1×TBST and once with 1×TBS. Chemiluminescence detection of the proteins was achieved using ECL Clarity (BioRad). Band intensities were analyzed using the ImageLab software from BioRad. For each sample, the intensity values of GAPDH bands were used to normalize the intensity measured for VEGFR1 and MBP.

shRNA Lentiviral Transduction

Primary rat OPC cultures were transduced using different MOIs (multiplicity of infection) of rat lentiviral particles that contained 4 unique 29mer shRNAs (OriGene) targeting the following sequences of VEGFR-1:

| (SEQ ID NO: 15) | |
|---|---|
| | 5′GGTCATAGAAGGAACCAATAAGACGGTTA′3, |
| (SEQ ID NO: 16) | |
| | 5′TCACTCTGAACCTTGTCATCAAGAATGTG′3, |
| (SEQ ID NO: 17) | |
| | 5′CTCGGTGACCTGCTTCAAGCCAATGTCCA′3, |
| (SEQ ID NO: 18) | |
| | 5′GAGCATCTATCAGGCAGCGGATTGACCAA′3. |

For RT-qPCR, transduction at MOI 8 and MOI 2 were used. Transfection efficiency was confirmed through GFP lentiviral expression. For immunocytochemistry, transfection with MOI 4 was used and labelling with Olig2 and MBP to quantify Olig2+MBP+ differentiated oligodendrocytes.

Pharmacological Treatment with VEGFR-1 Blocking Peptide.

Mouse cerebellar slices were prepared as previously described. Slices were treated with the basal medium alone or the basal medium containing specific VEGFR-1 blocking peptide (sequence: GNQWFI (SEQ ID NO: 6)), at a concentration of 10 μM, from 5 to 10 days in vitro. Slices were fixed and stained with Calbindin (CaBP) to stain Purkinje cells and MBP to label myelinated axons.

### Results

### VEGFR-1 Inhibition Enhances Neuroprotection in Organotypic Cerebellar Slices

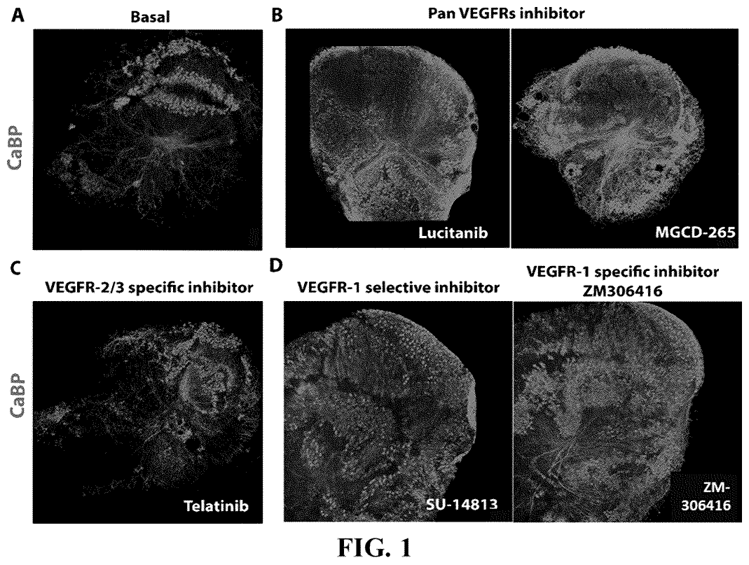

We tested the impact of VEGFRs subtype inhibitors on neuroprotection using an ex-vivo model of organotypic mouse cerebellar slice cultures, prepared from postnatal day 0 (P0) mouse cerebella. Interestingly, organotypic cerebellar slices treated during 10 days with the pan-VEGFR inhibitors Lucitanib or MGCD-265 (500 nM) (FIG. 1B) showed a higher number of Purkinje cells with respect to the basal condition (FIG. 1A), strongly arguing for a role of VEGFRs inhibitors on Purkinje cell survival. To determine whether a VEGFR subtype is selectively involved in the neuroprotection of Purkinje cells, we examined selective inhibitors of VEGFR-2/3 (Telatinib) and VEGFR-1 (SU14813 and ZM306416). We showed that the selective and specific inhibitors of VEGFR-1 SU14813 and ZM306416, respectively, but not of VEGFR-2/3 (Telatinib), enhance neuroprotection of Purkinje cells in organotypic cerebellar slices (FIGS. 1C and D), similarly to that observed with the pan-VEGFR inhibitors (Lucitanib or MGCD-265). Altogether, these data demonstrate that inhibition of VEGFR-1 enhances Purkinje cell survival and protect them from apoptosis in organotypic cerebellar slices. It is worth noting that Purkinje cells undergo a massive apoptosis from P1 to P5, during mouse cerebellar development.

### VEGFR-1 Inhibition Promotes Oligodendrocyte Differentiation and Myelination in Primary Cultures and Mouse Organotypic Cerebellar Slices

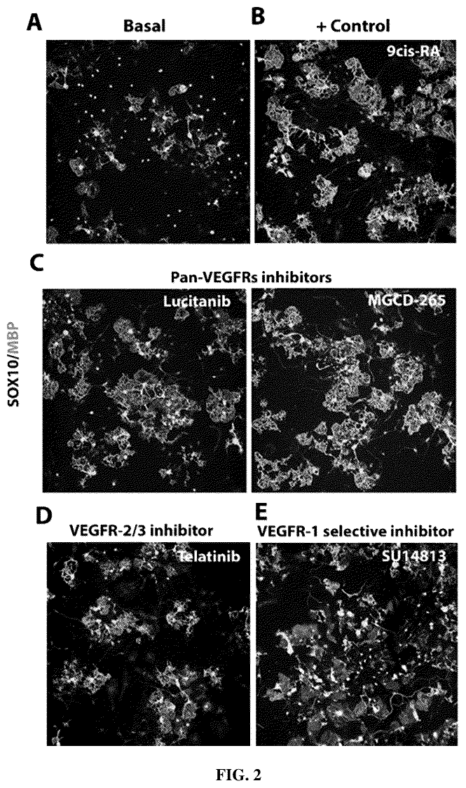

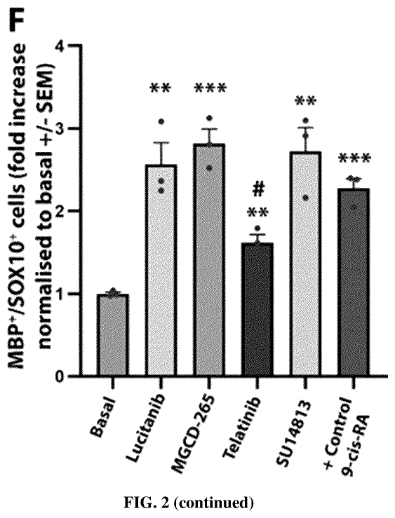

To investigate the role of VEGFR-1 in OPC differentiation and myelination using in vitro and ex-vivo models, we first analyzed effects of pan-VEGFRs and selective VEGFR-2/3 or VEGFR-1 inhibitors on OPC differentiation and maturation. The data are illustrated for Lucitanib and MGCD-265 (pan-VEGFR inhibitors), Telatinib (VEGFR-2/3 inhibitor) and SU14813 (VEGFR-1 inhibitor). Treatments of rat primary OPC cultures, with all compounds tested induced a significant increase of the number of SOX10⁺MBP⁺ differentiated oligodendrocytes, after 5 days in vitro (FIGS. 2A-F). Interestingly, the pan-VEGFR inhibitors and the VEGFR-1 inhibitor induce a stronger increase of OPC differentiation as compared to the VEGFR-2/3 inhibitor (FIG. 2F). To note, the dose response analysis at different concentrations (1 nM, 100 nM, 500 nM and 1 μM) showed a significant effect on OPC differentiation at 100 nM, with a peak at 500 nM for all compounds (data not shown).

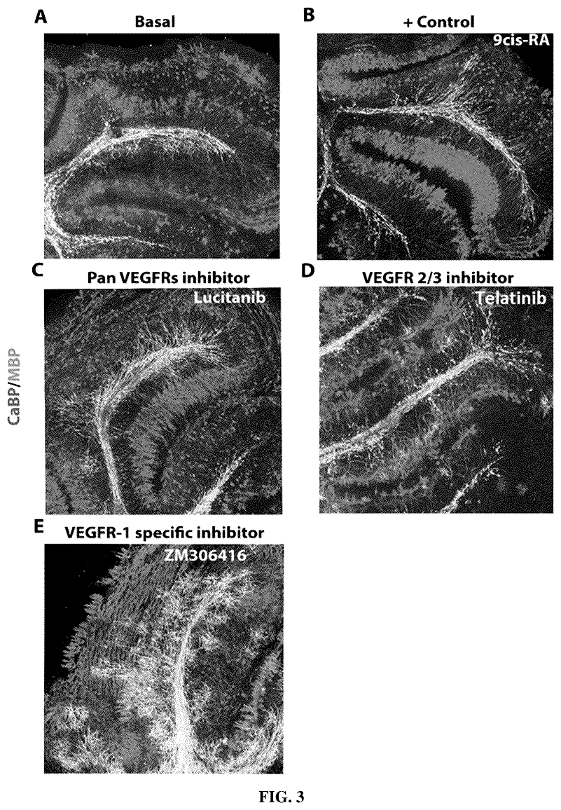

This latter concentration was next used for further validation of the effects of these compounds on myelination of organotypic cerebellar slices, prepared from P7 mouse brains. Slices were treated with the compounds for 3 days ex vivo. Remarkably, we found numerous MBP⁺ mature oligodendrocytes in organotypic cerebellar slices, treated with the pan-VEGFRs inhibitors (Lucitanib or MGCD 265) or with the VEGFR-1 selective and specific inhibitors (SU14813 and ZM306416) (FIGS. 3A-E). Moreover, quantification of myelination index showed that the density of myelinated axons was higher in cerebellar slices treated with these two VEGFR-1 inhibitor compounds family with respect to the basal condition, with respect to the positive control 9 cis-RA, with respect to the VEGFR-2/3 inhibitor and, with respect to the pan-VEGFR inhibitors (FIG. 3F). These data clearly demonstrated a significant increase of the density of myelinated axons after treatment with VEGFR-1 specific inhibitors, and importantly, show that VEGFR-1 inhibitors are more efficient than pan-VEGFR inhibitors or VEGFR-2/3 inhibitors for inducing myelination.

### VEGFR-1 Expression Decreases During Oligodendrocyte Differentiation In Vitro

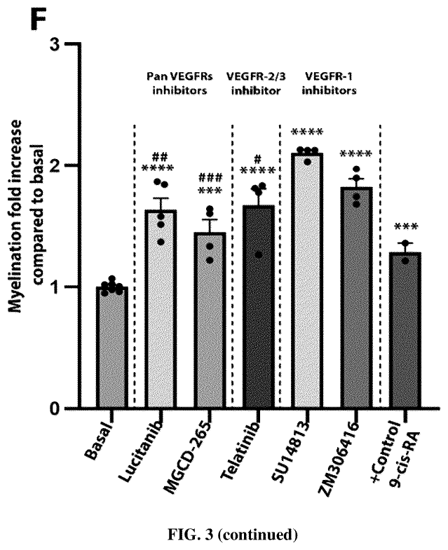

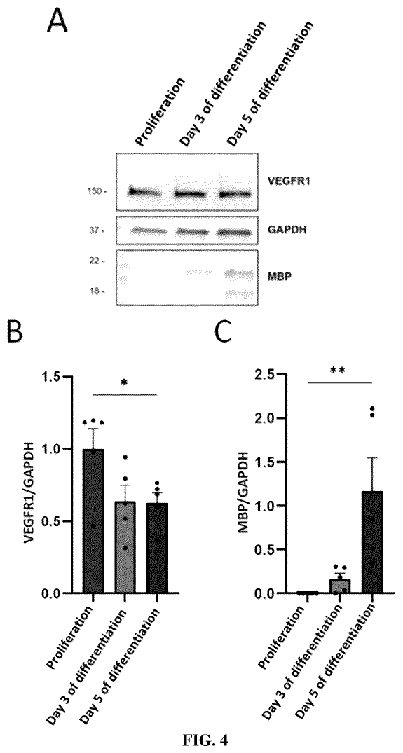

To validate the expression of VEGFR-1 during oligodendrocyte differentiation and maturation, we investigated changes of VEGFR-1 protein expression in rat primary OPC cultures. To do so, cells were lysed in proliferation condition, at D3 and D5 (3 and 5 days in differentiation medium), for protein extractions. Western Blots analysis revealed a decrease in expression of 37% of the full length VEGFR-1 at D3 and D5 compared to proliferation (FIGS. 4A, B), indicating that VEGFR-1 expression decreases during oligodendrocyte differentiation. MBP protein levels at D5 with respect to D3 and proliferation condition confirmed the increase in differentiated mature oligodendrocytes in cultures (FIG. 4C). Our data indicate that VEGFR-1 is expressed by oligodendroglial cells, and predominantly at the OPC stage. Interestingly, we also found that VEGFR-1 expression is also downregulated during OPC differentiation and maturation.

Knock-Down of VEGFR-1 Using shRNA Lentiviral Transduction or VEGFR1-Blocking Peptides Promote Oligodendrocyte Differentiation

To determine the functional role of VEGFR-1 in oligodendroglial lineage progression and myelination, we first used a shRNA-mediated knock-down strategy of VEGFR-1 in primary OPC cultures. Lentiviruses expressing different shRNAs that target VEGFR-1 were used to transfect primary rat OPC cultures. Out of the 4 shRNAs tested, 3 target exons 11-12, exons 12-13, and exon 7 coding for the immunoglobulin-like loops of the full-length and soluble form, and 1 targets exon 26 coding for the tyrosine kinase domain of the full-length form. A scrambled shRNA was used as a control.

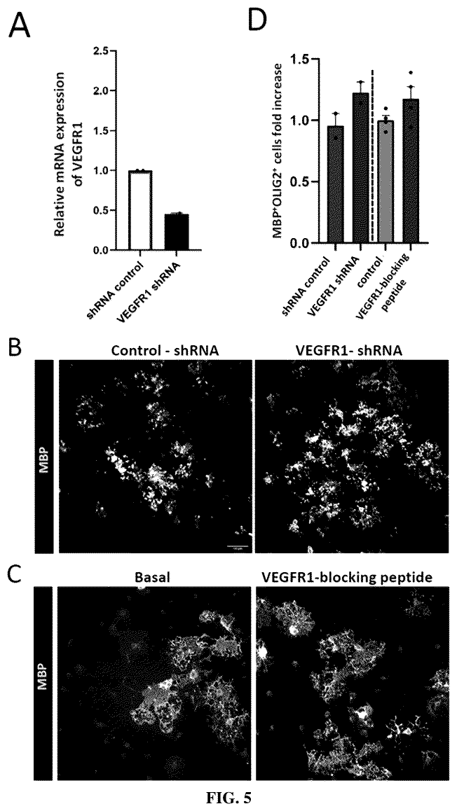

Our data confirmed efficient lentivirus transduction with multiplicity of infection 4 (MOI 4) and knock-down of VEGFR-1 (FIG. 5A). RT-qPCR also revealed an increase in expression of MOG (myelin oligodendrocyte glycoprotein), which is expressed mainly by mature myelinating oligodendrocytes (data not shown). This result suggests that VEGFR-1 knock-down in oligodendroglia promotes their differentiation. To further confirm the effect of VEGFR-1 inhibition on OPC differentiation, we used shRNA or a VEGFR-1 specific blocking peptide (sequence: GNQWFI) that binds to the ligand-binding site of this receptor, thus limiting the ligand-receptor interactions on rat primary OPC cultures. Immunolabeling was performed on VEGFR-1-shRNA and VEGFR-1-blocking peptide treated cultures and compared to shRNA-control and control conditions, respectively. At 5 days in differentiation medium, cells were immunolabeled with Olig2 and MBP to assess the proportion of differentiated oligodendrocytes (FIGS. 5B, C and D). To do so, we quantified Olig2+MBP+ differentiated oligodendrocytes over the total Olig2+ oligodendroglial cells (ratio of Olig2+MBP+ cells/Olig2+ cells) for each condition. This showed a 1.23±0.12-fold increase and a 1.18±0.19-fold increase in the number of Olig2+MBP+ cells when transfected with VEGFR-1-specific shRNA or treated with VEGFR-1-blocking peptide compared to controls (FIG. 5D). These results indicate that inhibiting VEGFR-1 promotes OPC differentiation into MBP+ oligodendrocytes.

### VEGFR-1 Blocking Peptides Promote Oligodendrocyte Maturation and Myelination

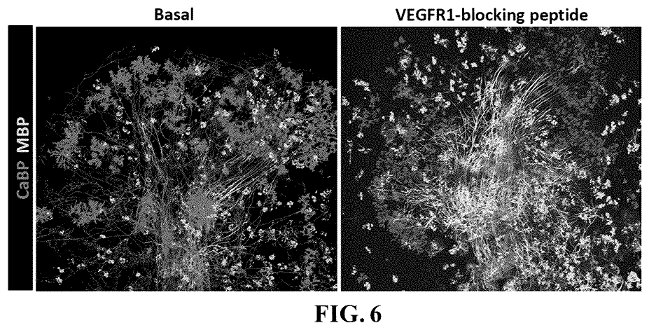

To further assess the effect of VEGFR1 inhibition on oligodendrocyte maturation and myelination, we used mouse organotypic cerebellar slices treated or untreated with the VEGFR-1 blocking peptide (sequence: GNQWFI). We showed that inhibition of VEGFR-1 with 10 μM of VEGFR-1-blocking peptide enhances ex-vivo myelination, in organotypic cerebellar slices (FIG. 6).

Altogether, these results strongly support an emerging role for VEGFR-1 in oligodendrocyte differentiation, myelination, and neuroprotection, and shed light on VEGFR-1 as a new pharmacological target for remyelination therapeutics.

1-15. (canceled)

16. A method for promoting myelination and/or neuroprotection in a subject in need thereof, comprising administering to the subject:
an inhibitor of vascular endothelial growth factor receptor 1 (VEGFR-1), or
a pharmaceutical composition comprising said inhibitor of VEGFR-1 and at least one pharmaceutically acceptable excipient.

17. The method according to claim 16, wherein said inhibitor is selected from the group consisting of small organic molecules, peptides, antibodies, antibody fragments, antibody mimetics, and nucleic acids.

18. The method according to claim 17, wherein said inhibitor is a small organic molecule.

19. The method according to claim 18, wherein said inhibitor is a small organic molecule selected from the group consisting of ZM306416 (CB 676475), SU14813, and salts, derivatives and combinations thereof.

20. The method according to claim 17, wherein said inhibitor is a peptide.

21. The method according to claim 20, wherein said inhibitor is a peptide selected from the group consisting of peptides with SEQ ID NOs: 6 to 10 and combinations thereof.

22. The method according to claim 21, wherein said inhibitor is a peptide with SEQ ID NO: 6.

23. The method according to claim 17, wherein said inhibitor is a nucleic acid.

24. The method according to claim 23, wherein said inhibitor is a shRNA selected from the group consisting of shRNAs with SEQ ID NOs: 15 to 18, and combinations thereof.

25. The method according to claim 16, wherein the subject is affected or diagnosed with a demyelinating disease.

26. The method according to claim 25, wherein the demyelinating disease is selected from the group consisting of multiple sclerosis, optic neuritis, cerebral ischemia, leukodystrophies and traumatic brain injury (TBI).

27. The method according to claim 26, wherein the demyelinating disease is multiple sclerosis.

28. The method according to claim 27, wherein the multiple sclerosis is selected from the group consisting of clinically isolated syndrome (CIS), relapsing remitting multiple sclerosis (RRMS), primary progressive multiple sclerosis (PPMS), and secondary progressive multiple sclerosis (SPMS),

29. The method according to claim 28, wherein the multiple sclerosis is a progressive form of the disease.

30. The method according to claim 16, said method further comprising administering to the subject an anti-inflammatory drug.

31. The method according to claim 16, wherein said subject has received, is receiving or will receive an anti-inflammatory drug.

32. The method according to claim 31, wherein the anti-inflammatory drug is an immunomodulator or an immunosuppressant.

33. The method according to claim 31, wherein the anti-inflammatory drug is selected from the group consisting of interferon beta, Glatiramer acetate, Fingolimod, Dimethyl fumarate, Diroximel fumarate, Teriflunomide, Siponimod, Cladribine, Natalizumab, Ocrelizumab, Alemtuzumab, Cyclophosphamide, Azathioprine, Mitoxandrone and combinations thereof.

34. A kit of parts comprising, in one part, at least one inhibitor of VEGFR-1 and, in a second part, at least one anti-inflammatory drug.

35. The kit of parts according to claim 34, wherein said inhibitor is a small organic molecule selected from the group consisting of ZM306416 (CB 676475), SU14813, and salts, derivatives and combinations thereof.


SEQUENCE LISTING (18 sequences)

SEQ ID NO: 1 - protein, 1338 aa, Homo sapiens
  REGION 1..1338: note = Human VEGFR-1
MVSYWDTGVLLCALLSCLLLTGSSSGSKLKDPELSLKGTQHIMQAGQTLHLQCRGEAAHKWSLPEMVSKESERLSITKSACGRNGKQFCSTLTLNTAQANHTGFYSCKYLAVPTSKKKETESAIYIFISDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVQISTPRPVKLLRGHTLVLNCTATTPLNTRVQMTWSYPDEKNKRASVRRRIDQSNSHANIFYSVLTIDKMQNKDKGLYTCRVRSGPSFKSVNTSVHIYDKAFITVKHRKQQVLETVAGKRSYRLSMKVKAFPSPEVVWLKDGLPATEKSARYLTRGYSLIIKDVTEEDAGNYTILLSIKQSNVFKNLTATLIVNVKPQIYEKAVSSFPDPALYPLGSRQILTCTAYGIPQPTIKWFWHPCNHNHSEARCDFCSNNEESFILDADSNMGNRIESITQRMAIIEGKNKMASTLVVADSRISGIYICIASNKVGTVGRNISFYITDVPNGFHVNLEKMPTEGEDLKLSCTVNKFLYRDVTWILLRTVNNRTMHYSISKQKMAITKEHSITLNLTIMNVSLQDSGTYACRARNVYTGEEILQKKEITIRDQEAPYLLRNLSDHTVAISSSTTLDCHANGVPEPQITWFKNNHKIQQEPGIILGPGSSTLFIERVTEEDEGVYHCKATNQKGSVESSAYLTVQGTSDKSNLELITLTCTCVAATLFWLLLTLFIRKMKRSSSEIKTDYLSIIMDPDEVPLDEQCERLPYDASKWEFARERLKLGKSLGRGAFGKVVQASAFGIKKSPTCRTVAVKMLKEGATASEYKALMTELKILTHIGHHLNVVNLLGACTKQGGPLMVIVEYCKYGNLSNYLKSKRDLFFLNKDAALHMEPKKEKMEPGLEQGKKPRLDSVTSSESFASSGFQEDKSLSDVEEEEDSDGFYKEPITMEDLISYSFQVARGMEFLSSRKCIHRDLAARNILLSENNVVKICDFGLARDIYKNPDYVRKGDTRLPLKWMAPESIFDKIYSTKSDVWSYGVLLWEIFSLGGSPYPGVQMDEDFCSRLREGMRMRAPEYSTPEIYQIMLDCWHRDPKERPRFAELVEKLGDLLQANVQQDGKDYIPINAILTGNSGFTYSTPAFSEDFFKESISAPKFNSGSSDDVRYVNAFKFMSLERIKTFEELLPNATSMFDDYQGDSSTLLASPMLKRFTWTDSKPKASLKIDLRVTSKSKESGLSDVSRPSFCHSSCGHVSEGKRRFTYDHAELERKIACCSPPPDYNSVVLYSTPPI

SEQ ID NO: 2 - protein, 456 aa, synthetic construct
  REGION 1..456: note = Heavy chain of icrucumab
QAQVVESGGGVVQSGRSLRLSCAASGFAFSSYGMHWVRQAPGKGLEWVAVIWYDGSNKYYADSVRGRFTISRDNSENTLYLQMNSLRAEDTAVYYCARDHYGSGVHHYFYYGLDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3 - protein, 215 aa, synthetic construct
  REGION 1..215: note = Light chain of icrucumab
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 4 - protein, 130 aa, synthetic construct
  REGION 1..130: note = Heavy chain variable region of D16F7
MGWSSIILFLVATASGVHSQYQQQVGSVWKPGASYKLSCKASGYWTNYWMHWYKQRPGQGLEWIGEIYPRNGNTNYDEKFKGKATLTIDTSSSTAYMQLSSLTSEDSAVYYCATYLGDYWGQGTTLTVSS

SEQ ID NO: 5 - protein, 131 aa, synthetic construct
  REGION 1..131: note = Light chain variable region of D16F7
MKLPVRLLVLMFWIPASSSDVLMTQTPLSLPVSLGDQASISCRSSQSIVHSNGNTYLEWFLQKPGQSPKLLIYKVSNRFSGIPDRFSGSGSGTDFTLKISRVEAEDLGVYFCFQGSHVPYTFGGGTKLEIK

SEQ ID NO: 6 - protein, 6 aa, synthetic construct
  REGION 1..6: note = Peptide
GNQWFI

SEQ ID NO: 7 - protein, 5 aa, synthetic construct
  REGION 1..5: note = Peptide
CLPRC

SEQ ID NO: 8 - protein, 12 aa, synthetic construct
  REGION 1..12: note = Peptide
WHSDMEWWYLLG

SEQ ID NO: 9 - protein, 13 aa, synthetic construct
  REGION 1..13: note = Peptide
TEGRELVIPARVT

SEQ ID NO: 10 - protein, 7 aa, synthetic construct
  REGION 1..7: note = Peptide
IPARVTS

SEQ ID NO: 11 - RNA, 21 nt, synthetic construct
  modified_base 20..21: mod_base = OTHER; note = unpaired deoxythymidine
  misc_feature 1..21: note = siRNA
  source 1..21: mol_type = other RNA
ggccagcacataggagagann

SEQ ID NO: 12 - RNA, 21 nt, synthetic construct
  modified_base 20..21: mod_base = OTHER; note = unpaired deoxythymidine
  misc_feature 1..21: note = siRNA
  source 1..21: mol_type = other RNA
tctctcctatgtgctggccnn

SEQ ID NO: 13 - RNA, 23 nt, synthetic construct
  modified_base 21..22: mod_base = OTHER; note = unpaired deoxythymidine
  misc_feature 1..23: note = siRNA
  source 1..23: mol_type = other RNA
  modified_base 1: mod_base = OTHER; note = 2'-deoxy abasic nucleotide
  modified_base 23: mod_base = OTHER; note = 2'-deoxy abasic nucleotide
nctgagtttaaaaggcacccnnn

SEQ ID NO: 14 - RNA, 21 nt, synthetic construct
  misc_feature 1..21: note = siRNA
  source 1..21: mol_type = other RNA
  modified_base 20: mod_base = OTHER; note = unpaired deoxythymidine
  modified_base 21: mod_base = OTHER; note = unpaired deoxythymidine
  modified_base 20^21: mod_base = OTHER; note = one phosphorothioate linkage
gggtgccttttaaactcagnn

SEQ ID NO: 15 - RNA, 29 nt, synthetic construct
  source 1..29: mol_type = other RNA
  misc_feature 1..29: note = shRNA
ggtcatagaaggaaccaataagacggtta

SEQ ID NO: 16 - RNA, 29 nt, synthetic construct
  source 1..29: mol_type = other RNA
  misc_feature 1..29: note = shRNA
tcactctgaaccttgtcatcaagaatgtg

SEQ ID NO: 17 - RNA, 29 nt, synthetic construct
  source 1..29: mol_type = other RNA
  misc_feature 1..29: note = shRNA
ctcggtgacctgcttcaagccaatgtcca

SEQ ID NO: 18 - RNA, 29 nt, synthetic construct
  source 1..29: mol_type = other RNA
  misc_feature 1..29: note = shRNA
gagcatctatcaggcagcggattgaccaa